(19) United States
(12) Patent Application Publication
(10) Pub. No.: US 2014/0303001 A1
(43) Pub. Date: Oct. 9, 2014

(54) BLADDER CANCER DETECTION AND MONITORING

(71) Applicant: Myriad Genetics, Inc., Salt Lake City, UT (US)

(72) Inventors: Stone Steve, Salt Lake City, UT (US); Michael Perry, Salt Lake City, UT (US); Alexander Gutin, Salt Lake City, UT (US)

(21) Appl. No.: 14/204,976

(22) Filed: Mar. 11, 2014

Related U.S. Application Data
(60) Provisional application No. 61/784,319, filed on Mar. 14, 2013.
(60) Provisional application No. 61/925,762, filed on Jan. 10, 2014.

(51) Int. Cl. C12Q 1/68

(52) U.S. Cl. CPC C12Q 1/6886
USPC 506/2; 435/6.11; 506/9

(57) ABSTRACT

Provided are molecular markers for detecting bladder cancer, especially recurrent bladder cancer, and methods of use thereof, including methods of monitoring for the recurrence of bladder cancer.

### RELATED APPLICATIONS

This application claims priority to U.S. provisional applications No. 61/784,319, filed Mar. 14, 2013, and 61/925,762, filed Jan. 10, 2014, the entire contents of which are hereby incorporated by reference.

### FIELD OF THE INVENTION

The present disclosure generally relates to molecular markers for detecting bladder cancer, especially recurrent bladder cancer, and methods of use thereof, including methods of monitoring for the recurrence of bladder cancer. The disclosure also relates to methods of treatment, automated methods of diagnosis, compositions, and kits for detecting the presence of genomic DNA from bladder cancer or tumor cells, including cell-free DNA that can be found in the urine of patients with bladder cancer, and patients with recurrent bladder cancer.

### TABLES

The instant application was filed with four (4) Tables (Tables A, B, C and D) under 37 C.F.R. §§1.52(e)(1)(iii) & 1.58(b), submitted electronically as the following tab-delimited text files:

a. TABLE A: MUTATION PANEL A
i. File name: 3350-01-2P-2014-01-10-TABLEA.txt
ii. Creation date: Mar. 14, 2013
iii. Size: 12,345 bytes

b. TABLE B: MUTATION PANEL B
i. File name: 3350-01-2P-2014-01-10-TABLEB.txt
ii. Creation date: Mar. 14, 2013
iii. Size: 342,622 bytes

c. TABLE C: MUTATION PANEL C
i. File name: 3350-01-2P-2014-01-10-TABLEC.txt
ii. Creation date: Mar. 14, 2013
iii. Size: 338,408 bytes

d. TABLE D: MUTATION PANEL D
i. File name: 3350-01-2P-2014-01-10-TABLED.txt
ii. Creation date: Jan. 7, 2014
iii. Size: 1,236,576 bytes
   Each of the above files and all of their contents are incorporated by reference herein in their entirety.

### BACKGROUND OF THE INVENTION

Cancer is a major public health problem, accounting for roughly 25% of all deaths in the United States. American Cancer Society, FACTS AND FIGURES. 2010. Though many treatments have been devised for various cancers, these treatments often vary in severity of side effects. It is useful for clinicians to be able to detect cancers early in their development, particularly through the use of non-invasive methods.

Urothelial cell carcinoma (UCC) of the urinary bladder is the 4th most common cancer in the United States, with an estimated 73,510 new cases and 14,880 deaths from bladder cancer in 2012, alone. American Cancer Society, FACTS AND FIGURES. 2012. Each year in the U.S. and EU, bladder cancer is diagnosed in >160,000 men and results in >48,000 deaths. While the five-year survival rate for early-stage bladder cancer is high, over 25% of patients present with advanced disease and around 70% experience recurrence or progression following treatment.

The prognosis for patients with progressive or recurrent invasive bladder cancer is generally poor. Management of recurrent cancers depends on the prior therapy used, site of recurrence, and patient-specific considerations. Early detection of primary bladder cancer, and especially recurrent bladder cancer, is important for disease management, and patient survival. Early detection of bladder cancers could potentially decrease morbidity of the disease, associated with current diagnostic and surveillance practices. Kelly et al., PLoS ONE 7(7):1-9 e40305, 2012.

Urinary cytology and cystoscopy are the current standard-of-care for bladder cancer detection and surveillance. Cystoscopy is the standard method of bladder tumor detection and/or confirmation; however it is an invasive, uncomfortable and costly procedure that results in urinary infection in up to 5% of cases. Almallah, et al., Urology 56:37-39 (2000). Urinary cytology is widely used for detection of bladder cancers, and has the advantage of up to 100% specificity. Unfortunately, urinary cytology is limited by its sensitivity, which is especially poor for low-grade bladder tumors.

Given the limitations of current methodologies, there is a clear need for more sensitive methods for the detection of bladder cancer, as well as recurrent bladder cancer, ideally using non-invasive procedures that utilize tumor-specific biomarkers of high specificity. Such tests have the potential to improve management of the disease and patient survival.

### SUMMARY OF THE INVENTION

Bladder cancer-specific mutations were identified by sequencing and analyzing the “exomes” (e.g., coding regions of the genomes) of over 40 human bladder cancer tumors in comparison to reference human genomes. The initially-identified discrepancies were limited to those not found in a database of human single nucleotide polymorphisms. The resulting reduced set of variants were analyzed for the effect that each change was predicted to have on the encoded protein or mRNA transcript. Those changes that were predicted to cause amino acid substitutions (missense mutations), truncation of the encoded protein due to the creation of a stop codon (nonsense mutations) or a shift in the reading frame (insertions and deletions), or an alteration in mRNA splicing, were considered further, while synonymous mutations (which change the original codon into a synonymous codon encoding the same amino acid and therefore did not alter the amino acid sequence of the encoded protein) were ignored. The frequency of occurrence of multiple mutations within a particular gene was used to further refine the list of mutations, and calculations based upon the length of the gene in which the mutations occurred were used to calculate a gene weighting score that approximately reflects the probability of that gene having that many mutations as a result of random chance. This gene weighting score was used to identify those genes in which human bladder cancer-specific mutations occurred at a frequency substantially greater than that expected from random chance. The resulting lists of mutations include bladder cancer-specific signature mutations. These mutations and the genes in which these mutations occurred can be used in the various methods, systems, kits, etc. of the present disclosure as diagnostic genes harboring mutations diagnostic of bladder cancer.

The novel bladder cancer-specific signature mutations thus identified, and other mutations in the diagnostic genes in which they were found, comprise biomarkers that can be used for the detection of bladder cancer. These bladder cancer-specific signature mutations, and the genes in which they occur, are provided herein. Diagnostic tests based upon the presence of these signature mutations are also provided, as are methods and kits for testing for the presence of bladder cancer, and especially recurrent bladder cancer, in patients. These methods, tests, and kits are suited to detect bladder cancer non-invasively, by testing for the presence of the signature mutations in nucleic acids (e.g., DNA) derived from patient samples (e.g., urine).

Other features and advantages of the invention will be apparent from the following Detailed Description, the accompanying Tables, and from the Claims.

### DETAILED DESCRIPTION OF THE INVENTION

### Definitions

As used herein, the phrase “indicator of bladder cancer,” refers to a mutation in a diagnostic gene of the disclosure, as detected by the methods disclosed herein below. As such, the phrase “indicator of bladder cancer,” can refer to one of the mutations specifically listed in the Tables provided herewith. Additionally, the phrase “indicator of bladder cancer,” can refer to other activating or deactivating (depending on the gene) mutations in the diagnostic genes of the disclosure besides those listed in the Tables, or a mutation predicted to either activate or deactivate such gene.

In some embodiments described herein the phrase “indicator of bladder cancer,” can refer to missense mutations that alter the sequence of amino acids in the protein encoded by a signature gene of the disclosure by converting the original codon encoding a first amino acid to a mutant codon encoding a second amino acid that is different from the first amino acid.

In other embodiments described herein the phrase “indicator of bladder cancer,” can refer to mutations that result in truncations of the protein encoded by a diagnostic gene of the disclosure. Such “truncation inducing” mutations include “nonsense mutations” where a single base change converts an amino acid encoding codon to a stop codon. Other “truncation inducing” mutations include “frameshift mutations,” which are insertions or deletions of one, two, or more (typically a multiple of one or two but not three) nucleotides, that either alter the normal or native reading frame of the codons that make up the coding sequence of the mRNA transcript of a diagnostic gene of the disclosure, or result in insertions or deletions of one or more amino acids in the protein encoded by a diagnostic gene of the disclosure. These insertion or deletion mutations can result in truncations of the encoded protein since altered reading frames can contain stop codons that will be encountered by the translational machinery of the cell in advance of the native stop codon. Additionally, insertions or deletions that result in altered reading frames can result in a different sequence of amino acids being added to the carboxyl-terminus of a protein as a result of the translational machinery translating in a different reading frame before encountering a stop codon in this new frame.

In other embodiments described herein the phrase “indicator of bladder cancer,” can refer to mutations that adversely alter the splicing of exons, or the removal of introns, from the transcript transcribed from a diagnostic gene of the disclosure. Such mutations that alter splicing the splicing of transcripts can occur at or near one of the so-called “splice junctions” that are found at the boundaries of the encoded exons and the introns that separate them. Such mutations can cause alterations in the amino acid sequence in the protein encoded by a diagnostic gene of the disclosure. Alternatively, such mutations result in truncations of the encoded protein, because stop codons can occur in multiple reading frames.

The phrase “deleterious mutation” as used herein, refers to a mutation that results in altered structure, expression or activity of the protein encoded by one of the diagnostic genes of the disclosure such that the mutation promotes the development or progression of cancer. This may include a deactivating mutation (e.g., ARID1A C132T) that results in, e.g., truncation of the encoded protein, an altered amino acid sequence of the encoded protein that yields a protein with no or attenuated activity, alterations in the splicing of the transcript of one of the diagnostic genes of the disclosure, reduction in the in vivo stability of the encoded transcript that yields lower expression of the protein, etc. This may also include an activating mutation (e.g., TERT promoter mutations C228T and/or C250T) that results in, e.g., an altered promoter that yields increased (e.g., constitutive) expression of the encoded protein, an altered amino acid sequence of the encoded protein that yields a protein with enhanced (e.g., constitutive) activity, increase in the in vivo stability of the encoded transcript that yields higher expression of the protein, etc.

As used herein, the phrases “signature mutation,” “tumor-specific mutation,” or “tumor-specific signature mutation” specifically refer to a mutation in a diagnostic gene of the disclosure, as detected by the methods disclosed herein below. As such, the phrases “signature mutation,” “tumor-specific mutation,” or “tumor-specific signature mutation” refers to one of the mutations specifically identified in the Tables provided herewith.

In contrast, the term “biomarker,” when used in the context of describing mutations in the diagnostic genes of the present disclosure, is synonymous with the phrase “indicator of bladder cancer,” and refers not only to one of the mutations specifically identified in the Tables provided herewith, but also to other mutations (e.g., nonsense, frameshift, large rearrangement, missense mutations) in the diagnostic genes of the disclosure besides those mutations identified in the Tables provided herewith.

The term “diagnostic gene,” as used herein, refers to one of the genes identified by the methods disclosed herein as containing or “harboring” a “signature mutation,” “tumor-specific mutation,” or “tumor-specific signature mutation.” Such “diagnostic genes” are those genes identified in Tables 1-6 provided herewith, and in GENE LIST A, GENE LIST B, GENE LIST C, GENE LIST D, GENE LIST X, and GENE LIST Z as defined below.

As used herein, the term “diagnostic test panel” means a predetermined group of diagnostic genes to be used in the methods, systems or kits of the disclosure (e.g., for the detection of bladder cancer or for the monitoring of bladder cancer recurrence).

The term “bladder cancer,” as used herein, refers to all forms of malignancy of urinary bladder tissues, but particularly includes malignancies arising from the epithelial lining (e.g., the urothelium) of the urinary bladder.

The most common type of bladder cancer, which recapitulates the normal histology of the urothelium, is known as transitional cell carcinoma, or urothelial cell carcinoma. Bladder cancers are often staged according to the following criteria:

Stage I. Cancer at this stage occurs in the bladder's inner lining but has not invaded the muscular bladder wall. This stage is commonly referred to as T1.

Stage II. At this stage, cancer has invaded the bladder wall but is still confined to the bladder. This stage is commonly referred to as T2.

Stage III. At this stage the cancer cells have spread through the bladder wall to surrounding tissue, and may also have spread to the prostate in men or the uterus or vagina in women. This stage is commonly referred to as T3.

Stage 1V. Cancer cells, by this stage may have spread to the lymph nodes and other organs, such as the lungs, bones or liver. This stage is commonly referred to as T4.

The phrase “recurrent bladder cancer,” as used herein, refers to those forms of bladder cancer that have reoccurred following an intervention (initial or subsequent), including surgical interventions, intended to remove any existing bladder cancer. Often the surgical intervention utilized for early stage bladder cancers is transurethral resection, in which superficial tumors (those which have not entered the surrounding muscle layer) are removed by electrocautery. The removed material can then be used for pathological examination and subsequent staging of the cancer. Recurrent bladder cancers may also arise after any combination of treatment by surgical intervention, chemotherapy, immunotherapy, and radiation treatment, with the latter methods typically being used in stage II, III and IV bladder cancers. Recurrent bladder cancers may also arise after surgical removal of all or part of the urinary bladder (e.g., cystectomy), particularly with metastatic forms of bladder cancer.

As used herein, the term “gene length,” when used in reference to a diagnostic gene as disclosed herein, refers to the length, in basepairs, of that portion of the diagnostic gene over which the gene was evaluated for the presence of bladder cancer-specific signature mutations according to the methods described in the Examples below. As such, the “gene length” for a particular diagnostic gene includes the length of the exons of that diagnostic gene, plus 100 base pairs from each (e.g., 5′ and 3′) end of any intervening sequences (e.g., introns) within that diagnostic gene, plus 100 base pairs of the untranslated regions of the exons abutting the 5′-most and 3′-most ends of the coding region.

The term “sample” or “biological sample,” as used herein, is an amount of tissue or bodily fluid taken from a subject, such as a mammal, or any biomolecule derived therefrom. Biomolecules derived from a tissue or fluid include molecules present in such tissue or fluid and extracted therefrom as well as artificial counterparts synthesized based on such endogenous biomolecules. Non-limiting examples of artificial counterparts include PCR products using endogenous nucleic acids as templates (e.g., cDNA synthesized from mRNA, PCR amplification of genomic DNA, etc.). Non-limiting examples of bodily fluids include urine, blood, plasma, serum, semen, perspiration, tears, mucus, and tissue lystates. A “sample” or “biological sample” further refers to a homogenate, lysate, or extract prepared from a whole organism or a subset of its tissues, cells, or component parts, or a fraction or portion thereof A “sample” or “biological sample” can refer to non-cellular biological material, such as blood or urine. In an exemplary embodiment, the sample is urine.

The term “nucleic acid” refers to deoxyribunucleotides, e.g., DNA, or ribonucleotides, e.g., RNA, and polymers thereof in either single- or double-stranded form. The term encompasses a nucleic acid containing known nucleotide analogs or modified backbone residues or linkages, which can be synthetic, naturally occurring, or normaturally occurring, which can have similar binding properties as the reference nucleic acids, and which can be metabolized in a manner similar to the reference nucleotides.

The terms “recurrence” and “metastatic progression” are well-known in the art and are used herein according to their known meanings. Because the methods of the disclosure can predict or determine a patient's likelihood of each, unless specified otherwise, a reference herein to an embodiment involving one applies equally to the other. As an example, the meaning of “metastatic progression” may be cancer-type dependent, with metastatic progression in one form of bladder cancer meaning something different from metastatic progression in another form of bladder cancer. However, within each cancer-type and subtype “recurrence” and “metastatic progression” are clearly understood to those skilled in the art. Because predicting recurrence and predicting metastatic progression are prognostic endeavors, “predicting prognosis” will often be used herein to refer to either or both. In these cases, a “poor prognosis” will generally refer to an increased likelihood of recurrence, metastatic progression, or both.

The term “subject” refers to any animal (e.g., a mammal), including, but not limited to humans, non-human primates, rodents, etc. The term “patient” refers to a human subject.

### Identification of Tumor-Specific Mutations and Diagnostic Genes

A detailed description of the methods generally described in this section can be found in EXAMPLE A, EXAMPLE B, EXAMPLE C, and EXAMPLE D, below.

The genomic sequence of the coding regions (e.g., exomes) of bladder cancer tumors can obtained and analyzed as compared to a reference human genome. Discrepancies between the bladder cancer tumor exomes and a reference human genome can be identified. Such discrepancies can be assessed for reproducibility between sequence reads. The discrepancies that represent real differences between the tumor exome and the reference genome can be classified as variants. Such variants can be assessed for novelty by comparing the variants against a database of known single nucleotide polymorphisms. Variants found in the database can be excluded from further analysis. These resulting subsets of variants can be further analyzed for their effect on the encoded protein and mRNA transcript encoding the protein. Variants causing a truncation of the encoded protein through the introduction of stop codons (e.g., nonsense mutations) and frameshift mutations can be included in further analyses, as can be missense mutations if at least one nonsense mutation or frameshift mutation had been mapped to the same gene. Variations that resulted in the change of the codon that was initially present in the protein for a synonymous codon (e.g., synonymous mutations) can be excluded.

In EXAMPLE A, a list of genes harboring at least one “suspected deleterious” mutation was prepared, and the genes in the list were subjected to a weighting procedure (as described in EXAMPLE A), which can include, but is not limited to:

Weight(for a given gene)=((# of Unique Variantŝ2)/(# of Variantŝ2))*(Number of samples affected by all variants)/(Square root Length of the gene)

to place a greater value on genes bearing unique variants as compared to those genes bearing variants that occurred in multiple samples, to place a greater value on genes that were affected by a greater number of unique variants, and to give a lower value to longer genes, which would be expected to have a greater number of variations due to random chance. The top 40 genes so identified in Example A were then evaluated based on their known functions to produce a list of 10 diagnostic genes harboring tumor-specific mutations. These 10 diagnostic genes comprise GENE LIST A, which includes TP53, NUP188, MUC16, CCDC168, KDM6A, SPTAN1, MLL2, ERBB3, ARID1A, and RB1. The tumor-specific signature mutations that were observed in these diagnostic genes (e.g., MUTATION PANEL A) are disclosed in Example A, and further information about the diagnostic genes is provided in Table 1.

TABLE 1
Diagnostic genes bearing bladder cancer-
specific signature mutations

| Gene | | Entrez | Gene |
|---|---|---|---|
| # | Gene | Gene ID | Length* |
| 1 | TP53 | 7157 | 4858 |
| 2 | NUP188 | 23511 | 13570 |
| 3 | MUC16 | 94025 | 60267 |
| 4 | CCDC168 | 643677 | 22081 |
| 5 | KDM6A | 7403 | 11359 |
| 6 | SPTAN1 | 6709 | 18304 |
| 7 | MLL2 | 8085 | 27398 |
| 8 | ERBB3 | 2065 | 11108 |
| 9 | ARID1A | 8289 | 12201 |
| 10 | RB1 | 5925 | 9918 |

EXAMPLE B demonstrates how additional efforts can be taken to further refine the list of diagnostic genes and tumor-specific signature mutations. Following sequencing and the identification of discrepancies between tumor genomes and the reference human genome, and following removal of polymorphisms known from the database single nucleotide polymorphisms, remaining variant calls can be also compared to a dataset of polymorphisms identified in coding regions of genomic sequences. For example, EXAMPLE B indentified polymorphimsms in 105 unrelated human blood samples. In EXAMPLE B, if a variant call was shared by more than 2 of the 105 blood samples, it was excluded from further consideration. This additional step was taken to cull any additional germline variants known from these independent reference genomic sequences, as well as to remove any process-specific artifacts that might have been present in the data. As with EXAMPLE A, variants causing a truncation of the encoded protein through the introduction of stop codons (e.g., nonsense mutations) or frameshift mutations, as well as variants resulting in the alteration of a single amino acid (missense mutations) were included for further analyses. Variations that resulted in the change of the native codon for a synonymous codon (e.g., synonymous mutations) were excluded. In EXAMPLE B a different gene weighting procedure (described in detail in the examples section, below) was used to identify genes that had more mutations than would be expected by random chance, based upon their length. The weighting procedure that can be used for further analysis can include:

Weight(for a given gene)=(((p*l)̂n)/n!)*ê−(p*l)

### Where:

p=Probability of base being mutated, this was calculated based off of the observed mutation rate in this dataset.

l=Length of the gene.

n=number of unique variants.

e=Euler's number, ˜2.718.

To further reduce the likelihood of including spurious genes, the ratio of unique variants to total variants found in the same gene had to be greater than 0.3. Genes with a weighted score of less than 10⁻⁷ (1×E-7) were retained and comprise the 99 diagnostic genes in GENE LIST B, which is provided along with additional details about the genes in Table 2. GENE LIST B includes TP53, NUP188, XIRP2, PLCG2, KDM6A, CCDC168, KIAA1671, KPRP, OR5L2, SPTAN1, ERBB3, SRRM2, ARID1A, FOXM1, MUC16, ISG20L2, ZC3H7A, MYBPC2, AHNAK2, HSPBAP1, SYNE1, ZNF208, PLD1, SMC2, OR8I2, BTN2A2, MLL2, JMJD1C, SLC35G6, VCAN, VPS13D, VCX3B, ZNF705G, RBBP8, IGSF6, DOCKS, C9orf174, NPC1L1, PCDHGA9, ACTB, DNHD1, LYST, SCAF11, ZNF846, LOC100133128, DNAH17, DYNC1H1, ANK3, KIAA0100, STAG2, FLG, ZNF623, DCHS1, CARD6, KIF13A, HEATR1, MMP8, SCN9A, NLRP13, ZFHX4, ODZ3, TNP2, LOC653720, SPAG17, FAM75D1, UGT1A3, ABCA5, MFHAS1, CLCA4, PLXNA2, C2orf16, CEP95, ZNF217, HMCN1, UGGT1, CDRT15L2, FAT1, ZNF493, AKAP13, CDH13, CCL20, CPSF2, PSD4, FAM193A, XPOT, WWP1, GLDC, TNN, PDE4A, DNAJC10, COL12A1, NF1, ITGA8, NPHP3, SAMD4A, COL21A1, NCKAP1L, MUC5B, and PCLO. The tumor-specific signature mutations that were observed in these 99 diagnostic genes (e.g., MUTATION PANEL B) are disclosed in Example B, and further information about the diagnostic genes is provided in Table 2.

TABLE 2
Diagnostic genes bearing bladder cancer-specific signature mutations

| | | | | | | Samples | |
|---|---|---|---|---|---|---|---|
| | | | | Unique | Total | Affected | |
| Gene | | Entrez | Gene | Variant | Variant | by all | Weighted |
| # | Gene | Gene ID | Length* | Count | Counts | Variants | Score |
| 1 | TP53 | 7157 | 4858 | 12 | 13 | 12 | 1.55E−29 |
| 2 | NUP188 | 23511 | 13570 | 12 | 18 | 11 | 3.38E−24 |
| 3 | XIRP2 | 129446 | 15610 | 9 | 17 | 17 | 7.66E−17 |
| 4 | PLCG2 | 5336 | 10808 | 8 | 19 | 13 | 5.49E−16 |
| 5 | KDM6A | 7403 | 11359 | 8 | 8 | 8 | 8.15E−16 |
| 6 | CCDC168 | 643677 | 22081 | 9 | 9 | 9 | 1.69E−15 |
| 7 | KIAA1671 | 85379 | 12683 | 8 | 18 | 16 | 1.96E−15 |
| 8 | KPRP | 448834 | 2921 | 6 | 6 | 6 | 5.65E−15 |
| 9 | OR5L2 | 26338 | 1135 | 5 | 9 | 8 | 2.39E−14 |
| 10 | SPTAN1 | 6709 | 18304 | 8 | 8 | 5 | 3.60E−14 |
| 11 | ERBB3 | 2065 | 11108 | 7 | 7 | 6 | 1.13E−13 |
| 12 | SRRM2 | 23524 | 11953 | 7 | 7 | 5 | 1.89E−13 |
| 13 | ARID1A | 8289 | 12201 | 7 | 11 | 9 | 2.18E−13 |
| 14 | FOXM1 | 2305 | 5403 | 6 | 19 | 16 | 2.24E−13 |
| 15 | MUC16 | 94025 | 60267 | 10 | 18 | 15 | 3.14E−13 |
| 16 | ISG20L2 | 81875 | 2648 | 5 | 11 | 6 | 1.64E−12 |
| 17 | ZC3H7A | 29066 | 8286 | 6 | 6 | 2 | 2.88E−12 |
| 18 | MYBPC2 | 4606 | 8698 | 6 | 10 | 7 | 3.84E−12 |
| 19 | AHNAK2 | 113146 | 19572 | 7 | 22 | 20 | 5.77E−12 |
| 20 | HSPBAP1 | 79663 | 3455 | 5 | 15 | 15 | 6.18E−12 |
| 21 | SYNE1 | 23345 | 57157 | 9 | 15 | 14 | 7.56E−12 |
| 22 | ZNF208 | 7757 | 9884 | 6 | 11 | 9 | 8.23E−12 |
| 23 | PLD1 | 5337 | 10966 | 6 | 19 | 17 | 1.53E−11 |
| 24 | SMC2 | 10592 | 11297 | 6 | 6 | 3 | 1.82E−11 |
| 25 | OR8I2 | 120586 | 1132 | 4 | 7 | 5 | 2.40E−11 |
| 26 | BTN2A2 | 10385 | 5367 | 5 | 13 | 11 | 5.55E−11 |
| 27 | MLL2 | 8085 | 27398 | 7 | 7 | 6 | 5.87E−11 |
| 28 | JMJD1C | 221037 | 13810 | 6 | 6 | 5 | 6.02E−11 |
| 29 | SLC35G6 | 643664 | 1593 | 4 | 11 | 10 | 9.41E−11 |
| 30 | VCAN | 1462 | 15228 | 6 | 6 | 6 | 1.08E−10 |
| 31 | VPS13D | 55187 | 30041 | 7 | 7 | 5 | 1.11E−10 |
| 32 | VCX3B | 425054 | 1764 | 4 | 9 | 8 | 1.41E−10 |
| 33 | ZNF705G | 100131980 | 1920 | 4 | 4 | 4 | 1.98E−10 |
| 34 | RBBP8 | 5932 | 7416 | 5 | 5 | 5 | 2.77E−10 |
| 35 | IGSF6 | 10261 | 2222 | 4 | 5 | 5 | 3.55E−10 |
| 36 | DOCK9 | 23348 | 19186 | 6 | 6 | 4 | 4.23E−10 |
| 37 | C9orf174 | 100499483 | 354 | 3 | 4 | 4 | 6.01E−10 |
| 38 | NPC1L1 | 29881 | 8897 | 5 | 5 | 4 | 6.84E−10 |
| 39 | PCDHGA9 | 56107 | 2686 | 4 | 4 | 4 | 7.57E−10 |
| 40 | ACTB | 60 | 2747 | 4 | 4 | 2 | 8.28E−10 |
| 41 | DNHD1 | 144132 | 22844 | 6 | 13 | 12 | 1.19E−09 |
| 42 | LYST | 1130 | 23851 | 6 | 6 | 5 | 1.53E−09 |
| 43 | SCAF11 | 9169 | 10542 | 5 | 5 | 4 | 1.59E−09 |
| 44 | ZNF846 | 162993 | 3327 | 4 | 4 | 4 | 1.78E−09 |
| 45 | LOC100133128 | 100133128 | 620 | 3 | 5 | 5 | 3.23E−09 |
| 46 | DNAH17 | 8632 | 27298 | 6 | 6 | 6 | 3.39E−09 |
| 47 | DYNC1H1 | 1778 | 27420 | 6 | 7 | 6 | 3.48E−09 |
| 48 | ANK3 | 288 | 28629 | 6 | 6 | 5 | 4.48E−09 |
| 49 | KIAA0100 | 9703 | 13143 | 5 | 13 | 12 | 4.72E−09 |
| 50 | STAG2 | 10735 | 13183 | 5 | 5 | 4 | 4.80E−09 |
| 51 | FLG | 2312 | 13344 | 5 | 6 | 6 | 5.09E−09 |
| 52 | ZNF623 | 9831 | 4558 | 4 | 4 | 4 | 6.22E−09 |
| 53 | DCHS1 | 8642 | 14137 | 5 | 5 | 3 | 6.77E−09 |
| 54 | CARD6 | 84674 | 4694 | 4 | 6 | 6 | 7.00E−09 |
| 55 | KIF13A | 63971 | 14303 | 5 | 5 | 5 | 7.17E−09 |
| 56 | HEATR1 | 55127 | 14505 | 5 | 5 | 5 | 7.69E−09 |
| 57 | MMP8 | 4317 | 4933 | 4 | 4 | 3 | 8.53E−09 |
| 58 | SCN9A | 6335 | 15069 | 5 | 5 | 5 | 9.28E−09 |
| 59 | NLRP13 | 126204 | 5346 | 4 | 4 | 4 | 1.17E−08 |
| 60 | ZFHX4 | 79776 | 16147 | 5 | 6 | 6 | 1.31E−08 |
| 61 | ODZ3 | 55714 | 16176 | 5 | 5 | 4 | 1.32E−08 |
| 62 | TNP2 | 7142 | 995 | 3 | 7 | 5 | 1.33E−08 |
| 63 | LOC653720 | 653720 | 5565 | 4 | 4 | 2 | 1.38E−08 |
| 64 | SPAG17 | 200162 | 16454 | 5 | 5 | 5 | 1.43E−08 |
| 65 | FAM75D1 | 389763 | 5629 | 4 | 4 | 4 | 1.44E−08 |
| 66 | UGT1A3 | 54659 | 1066 | 3 | 3 | 3 | 1.64E−08 |
| 67 | ABCA5 | 23461 | 16948 | 5 | 5 | 5 | 1.66E−08 |
| 68 | MFHAS1 | 9258 | 5852 | 4 | 11 | 9 | 1.68E−08 |
| 69 | CLCA4 | 22802 | 5937 | 4 | 4 | 4 | 1.78E−08 |
| 70 | PLXNA2 | 5362 | 17765 | 5 | 5 | 4 | 2.09E−08 |
| 71 | C2orf16 | 84226 | 6400 | 4 | 4 | 4 | 2.40E−08 |
| 72 | CEP95 | 90799 | 6567 | 4 | 5 | 5 | 2.66E−08 |
| 73 | ZNF217 | 7764 | 6630 | 4 | 4 | 3 | 2.76E−08 |
| 74 | HMCN1 | 83872 | 39142 | 6 | 6 | 6 | 2.80E−08 |
| 75 | UGGT1 | 56886 | 19121 | 5 | 5 | 4 | 3.00E−08 |
| 76 | CDRT15L2 | 256223 | 1386 | 3 | 6 | 5 | 3.59E−08 |
| 77 | FAT1 | 2195 | 19980 | 5 | 5 | 5 | 3.72E−08 |
| 78 | ZNF493 | 284443 | 7280 | 4 | 6 | 3 | 4.00E−08 |
| 79 | AKAP13 | 11214 | 21025 | 5 | 5 | 4 | 4.78E−08 |
| 80 | CDH13 | 1012 | 7714 | 4 | 4 | 3 | 5.04E−08 |
| 81 | CCL20 | 6364 | 1638 | 3 | 3 | 2 | 5.92E−08 |
| 82 | CPSF2 | 53981 | 8135 | 4 | 4 | 4 | 6.22E−08 |
| 83 | PSD4 | 23550 | 8261 | 4 | 4 | 1 | 6.61E−08 |
| 84 | FAM193A | 8603 | 8372 | 4 | 4 | 3 | 6.97E−08 |
| 85 | XPOT | 11260 | 8684 | 4 | 4 | 2 | 8.06E−08 |
| 86 | WWP1 | 11059 | 8695 | 4 | 6 | 2 | 8.10E−08 |
| 87 | GLDC | 2731 | 8759 | 4 | 11 | 6 | 8.34E−08 |
| 88 | TNN | 63923 | 8789 | 4 | 12 | 11 | 8.45E−08 |
| 89 | PDE4A | 5141 | 8907 | 4 | 10 | 9 | 8.91E−08 |
| 90 | DNAJC10 | 54431 | 8969 | 4 | 4 | 3 | 9.16E−08 |
| 91 | COL12A1 | 1303 | 24090 | 5 | 5 | 5 | 9.32E−08 |
| 92 | NF1 | 4763 | 24550 | 5 | 13 | 7 | 1.02E−07 |
| 93 | ITGA8 | 8516 | 9231 | 4 | 4 | 4 | 1.03E−07 |
| 94 | NPHP3 | 27031 | 1978 | 3 | 6 | 6 | 1.04E−07 |
| 95 | SAMD4A | 23034 | 9419 | 4 | 8 | 8 | 1.11E−07 |
| 96 | COL21A1 | 81578 | 9746 | 4 | 4 | 4 | 1.27E−07 |
| 97 | NCKAP1L | 3071 | 9933 | 4 | 4 | 4 | 1.37E−07 |
| 98 | MUC5B | 727897 | 26870 | 5 | 16 | 13 | 1.59E−07 |
| 99 | PCLO | 27445 | 27278 | 5 | 5 | 5 | 1.71E−07 |

EXAMPLE C demonstrates how additional efforts can be taken to further refine the list of diagnostic genes and tumor-specific signature mutations. In EXAMPLE C, the same starting data and refined procedure as in EXAMPLE B was used, with one exception: Variants predicted to adversely affect the splicing of the encoded transcript can be also included for further analyses. Thus, genes bearing splice-site altering mutations, in addition to genes bearing missense mutations, nonsense mutations, insertions or deletions can be subjected to the same weighting procedure as described for EXAMPLE B. As with EXAMPLE B, genes with a weighted score of less than 10⁻⁷ (1×E-7) can be retained as diagnostic genes. These genes comprise the 99 diagnostic genes in GENE LIST C, which is provided along with additional details about the genes in Table 3. GENE LIST C, which substantially overlaps with GENE LIST B, includes TP53, NUP188, XIRP2, PLCG2, FOXM1, KDM6A, ARID1A, CCDC168, KIAA1671, KPRP, MUC16, OR5L2, SPTAN1, ERBB3, SRRM2, SNRNP200, ISG20L2, ZC3H7A, MYBPC2, AHNAK2, HSPBAP1, SYNE1, ZNF208, PLD1, SMC2, OR8I2, STAG2, BTN2A2, MLL2, JMJD1C, SLC35G6, VCAN, VPS13D, VCX3B, ZNF705G, RBBP8, IGSF6, DOCKS, C9orf174, NPC1L1, PCDHGA9, ACTB, DNHD1, LYST, SCAF11, ZNF846, NF1, CACNA2D3, LAPTM4B, LOC100133128, PCLO, DNAH17, DYNC1H1, ANK3, KIAA0100, FLG, ABCB5, POLR3C, ZNF623, DCHS1, CARD6, KIF13A, HEATR1, WDR6, MMP8, SCN9A, NLRP13, ZFHX4, ODZ3, TNP2, LOC653720, SPAG17, FAM75D1, UGT1A3, ABCA5, MFHAS1, CLCA4, CRTAC1, CHD6, PLXNA2, RYR2, C2orf16, CEP95, ZNF217, HMCN1, UGGT1, CDRT15L2, FAT1, ZNF493, AKAP13, CDH13, CCL20, CPSF2, PSD4, FAM193A, XPOT, WWP1, GLDC, and TNN. The tumor-specific signature mutations that were observed in these 99 diagnostic genes (e.g., MUTATION PANEL C) are disclosed in Example C, and further information about the diagnostic genes is provided in Table 3.

TABLE 3
Diagnostic genes bearing bladder cancer-specific signature mutations

| | | | | | | Samples | |
|---|---|---|---|---|---|---|---|
| | | | | Unique | Total | Affected | |
| Gene | | Entrez | Gene | Variant | Variant | by all | Weighted |
| # | Gene | Gene ID | Length* | Count | Counts | Variants | Score |
| 1 | TP53 | 7157 | 4858 | 13 | 14 | 13 | 2.52E−32 |
| 2 | NUP188 | 23511 | 13570 | 12 | 18 | 11 | 3.38E−24 |
| 3 | XIRP2 | 129446 | 15610 | 10 | 18 | 18 | 5.18E−19 |
| 4 | PLCG2 | 5336 | 10808 | 8 | 19 | 13 | 5.49E−16 |
| 5 | FOXM1 | 2305 | 5403 | 7 | 20 | 17 | 7.49E−16 |
| 6 | KDM6A | 7403 | 11359 | 8 | 8 | 8 | 8.15E−16 |
| 7 | ARID1A | 8289 | 12201 | 8 | 12 | 10 | 1.44E−15 |
| 8 | CCDC168 | 643677 | 22081 | 9 | 9 | 9 | 1.69E−15 |
| 9 | KIAA1671 | 85379 | 12683 | 8 | 18 | 16 | 1.96E−15 |
| 10 | KPRP | 448834 | 2921 | 6 | 6 | 6 | 5.65E−15 |
| 11 | MUC16 | 94025 | 60267 | 11 | 19 | 16 | 7.46E−15 |
| 12 | OR5L2 | 26338 | 1135 | 5 | 9 | 8 | 2.39E−14 |
| 13 | SPTAN1 | 6709 | 18304 | 8 | 8 | 5 | 3.60E−14 |
| 14 | ERBB3 | 2065 | 11108 | 7 | 7 | 6 | 1.13E−13 |
| 15 | SRRM2 | 23524 | 11953 | 7 | 7 | 5 | 1.89E−13 |
| 16 | SNRNP200 | 23020 | 15227 | 7 | 22 | 19 | 1.01E−12 |
| 17 | ISG20L2 | 81875 | 2648 | 5 | 11 | 6 | 1.64E−12 |
| 18 | ZC3H7A | 29066 | 8286 | 6 | 6 | 2 | 2.88E−12 |
| 19 | MYBPC2 | 4606 | 8698 | 6 | 10 | 7 | 3.84E−12 |
| 20 | AHNAK2 | 113146 | 19572 | 7 | 22 | 20 | 5.77E−12 |
| 21 | HSPBAP1 | 79663 | 3455 | 5 | 15 | 15 | 6.18E−12 |
| 22 | SYNE1 | 23345 | 57157 | 9 | 15 | 14 | 7.56E−12 |
| 23 | ZNF208 | 7757 | 9884 | 6 | 11 | 9 | 8.23E−12 |
| 24 | PLD1 | 5337 | 10966 | 6 | 19 | 17 | 1.53E−11 |
| 25 | SMC2 | 10592 | 11297 | 6 | 6 | 3 | 1.82E−11 |
| 26 | OR8I2 | 120586 | 1132 | 4 | 7 | 5 | 2.40E−11 |
| 27 | STAG2 | 10735 | 13183 | 6 | 6 | 5 | 4.57E−11 |
| 28 | BTN2A2 | 10385 | 5367 | 5 | 13 | 11 | 5.55E−11 |
| 29 | MLL2 | 8085 | 27398 | 7 | 7 | 6 | 5.87E−11 |
| 30 | JMJD1C | 221037 | 13810 | 6 | 6 | 5 | 6.02E−11 |
| 31 | SLC35G6 | 643664 | 1593 | 4 | 11 | 10 | 9.41E−11 |
| 32 | VCAN | 1462 | 15228 | 6 | 6 | 6 | 1.08E−10 |
| 33 | VPS13D | 55187 | 30041 | 7 | 7 | 5 | 1.11E−10 |
| 34 | VCX3B | 425054 | 1764 | 4 | 9 | 8 | 1.41E−10 |
| 35 | ZNF705G | 100131980 | 1920 | 4 | 4 | 4 | 1.98E−10 |
| 36 | RBBP8 | 5932 | 7416 | 5 | 5 | 5 | 2.77E−10 |
| 37 | IGSF6 | 10261 | 2222 | 4 | 5 | 5 | 3.55E−10 |
| 38 | DOCK9 | 23348 | 19186 | 6 | 6 | 4 | 4.23E−10 |
| 39 | C9orf174 | 100499483 | 354 | 3 | 4 | 4 | 6.01E−10 |
| 40 | NPC1L1 | 29881 | 8897 | 5 | 5 | 4 | 6.84E−10 |
| 41 | PCDHGA9 | 56107 | 2686 | 4 | 4 | 4 | 7.57E−10 |
| 42 | ACTB | 60 | 2747 | 4 | 4 | 2 | 8.28E−10 |
| 43 | DNHD1 | 144132 | 22844 | 6 | 13 | 12 | 1.19E−09 |
| 44 | LYST | 1130 | 23851 | 6 | 6 | 5 | 1.53E−09 |
| 45 | SCAF11 | 9169 | 10542 | 5 | 5 | 4 | 1.59E−09 |
| 46 | ZNF846 | 162993 | 3327 | 4 | 4 | 4 | 1.78E−09 |
| 47 | NF1 | 4763 | 24550 | 6 | 18 | 10 | 1.81E−09 |
| 48 | CACNA2D3 | 55799 | 11097 | 5 | 6 | 5 | 2.05E−09 |
| 49 | LAPTM4B | 55353 | 3631 | 4 | 4 | 1 | 2.52E−09 |
| 50 | LOC100133128 | 100133128 | 620 | 3 | 5 | 5 | 3.23E−09 |
| 51 | PCLO | 27445 | 27278 | 6 | 6 | 6 | 3.37E−09 |
| 52 | DNAH17 | 8632 | 27298 | 6 | 6 | 6 | 3.39E−09 |
| 53 | DYNC1H1 | 1778 | 27420 | 6 | 7 | 6 | 3.48E−09 |
| 54 | ANK3 | 288 | 28629 | 6 | 6 | 5 | 4.48E−09 |
| 55 | KIAA0100 | 9703 | 13143 | 5 | 13 | 12 | 4.72E−09 |
| 56 | FLG | 2312 | 13344 | 5 | 6 | 6 | 5.09E−09 |
| 57 | ABCB5 | 340273 | 13391 | 5 | 5 | 5 | 5.18E−09 |
| 58 | POLR3C | 10623 | 4546 | 4 | 4 | 4 | 6.16E−09 |
| 59 | ZNF623 | 9831 | 4558 | 4 | 4 | 4 | 6.22E−09 |
| 60 | DCHS1 | 8642 | 14137 | 5 | 5 | 3 | 6.77E−09 |
| 61 | CARD6 | 84674 | 4694 | 4 | 6 | 6 | 7.00E−09 |
| 62 | KIF13A | 63971 | 14303 | 5 | 5 | 5 | 7.17E−09 |
| 63 | HEATR1 | 55127 | 14505 | 5 | 5 | 5 | 7.69E−09 |
| 64 | WDR6 | 11180 | 4895 | 4 | 6 | 6 | 8.27E−09 |
| 65 | MMP8 | 4317 | 4933 | 4 | 4 | 3 | 8.53E−09 |
| 66 | SCN9A | 6335 | 15069 | 5 | 5 | 5 | 9.28E−09 |
| 67 | NLRP13 | 126204 | 5346 | 4 | 4 | 4 | 1.17E−08 |
| 68 | ZFHX4 | 79776 | 16147 | 5 | 6 | 6 | 1.31E−08 |
| 69 | ODZ3 | 55714 | 16176 | 5 | 5 | 4 | 1.32E−08 |
| 70 | TNP2 | 7142 | 995 | 3 | 7 | 5 | 1.33E−08 |
| 71 | LOC653720 | 653720 | 5565 | 4 | 4 | 2 | 1.38E−08 |
| 72 | SPAG17 | 200162 | 16454 | 5 | 5 | 5 | 1.43E−08 |
| 73 | FAM75D1 | 389763 | 5629 | 4 | 4 | 4 | 1.44E−08 |
| 74 | UGT1A3 | 54659 | 1066 | 3 | 3 | 3 | 1.64E−08 |
| 75 | ABCA5 | 23461 | 16948 | 5 | 5 | 5 | 1.66E−08 |
| 76 | MFHAS1 | 9258 | 5852 | 4 | 11 | 9 | 1.68E−08 |
| 77 | CLCA4 | 22802 | 5937 | 4 | 4 | 4 | 1.78E−08 |
| 78 | CRTAC1 | 55118 | 5948 | 4 | 6 | 6 | 1.79E−08 |
| 79 | CHD6 | 84181 | 17737 | 5 | 7 | 5 | 2.07E−08 |
| 80 | PLXNA2 | 5362 | 17765 | 5 | 5 | 4 | 2.09E−08 |
| 81 | RYR2 | 6262 | 37260 | 6 | 6 | 5 | 2.10E−08 |
| 82 | C2orf16 | 84226 | 6400 | 4 | 4 | 4 | 2.40E−08 |
| 83 | CEP95 | 90799 | 6567 | 4 | 5 | 5 | 2.66E−08 |
| 84 | ZNF217 | 7764 | 6630 | 4 | 4 | 3 | 2.76E−08 |
| 85 | HMCN1 | 83872 | 39142 | 6 | 6 | 6 | 2.80E−08 |
| 86 | UGGT1 | 56886 | 19121 | 5 | 5 | 4 | 3.00E−08 |
| 87 | CDRT15L2 | 256223 | 1386 | 3 | 6 | 5 | 3.59E−08 |
| 88 | FAT1 | 2195 | 19980 | 5 | 5 | 5 | 3.72E−08 |
| 89 | ZNF493 | 284443 | 7280 | 4 | 6 | 3 | 4.00E−08 |
| 90 | AKAP13 | 11214 | 21025 | 5 | 5 | 4 | 4.78E−08 |
| 91 | CDH13 | 1012 | 7714 | 4 | 4 | 3 | 5.04E−08 |
| 92 | CCL20 | 6364 | 1638 | 3 | 3 | 2 | 5.92E−08 |
| 93 | CPSF2 | 53981 | 8135 | 4 | 4 | 4 | 6.22E−08 |
| 94 | PSD4 | 23550 | 8261 | 4 | 4 | 1 | 6.61E−08 |
| 95 | FAM193A | 8603 | 8372 | 4 | 4 | 3 | 6.97E−08 |
| 96 | XPOT | 11260 | 8684 | 4 | 4 | 2 | 8.06E−08 |
| 97 | WWP1 | 11059 | 8695 | 4 | 6 | 2 | 8.10E−08 |
| 98 | GLDC | 2731 | 8759 | 4 | 11 | 6 | 8.34E−08 |
| 99 | TNN | 63923 | 8789 | 4 | 12 | 11 | 8.45E−08 |

Also provided is a 61-member subset of the genes of GENE LIST C, known as GENE LIST X. GENE LIST X comprises AHNAK2, AKAP13, BTN2A2, CARD6, CCL20, CLCA4, COL12A1, COL21A1, CPSF2, DCHS1, DNAH17, DNAJC10, DOCKS, DYNC1H1, FAM193A, FLG, GLDC, HMCN1, HSPBAP1, IGSF6, ISG20L2, ITGA8, JMJD1C, KDM6A, KIAA0100, KIAA1671, KPRP, LYST, MFHAS1, MLL2, MYBPC2, NCKAP1L, NPC1L1, NPHP3, NUP188, ODZ3, PCLO, PDE4A, PLCG2, PLXNA2, PSD4, RBBP8, SAMD4A, SCN9A, SMC2, SNRNP200, SPAG17, STAG2, SYNE1, TNP2, UGGT1, UGT1A3, VCX3B, VPS13D, WDR6, XIRP2, XPOT, ZC3H7A, ZFHX4, ZNF208, and ZNF493. The bladder cancer-specific signature mutations identified in the genes of GENE LIST X are provided within MUTATION PANELS B and C. GENE LIST X is shown in Table 4.

TABLE 4
Diagnostic genes bearing bladder cancer-
specific signature mutations

| | Entrez | Gene |
|---|---|---|
| Gene | Gene ID | Length* |
| AHNAK2 | 113146 | 19572 |
| AKAP13 | 11214 | 21025 |
| BTN2A2 | 10385 | 5367 |
| CARD6 | 84674 | 4694 |
| CCL20 | 6364 | 1638 |
| CLCA4 | 22802 | 5937 |
| COL12A1 | 81578 | 24090 |
| COL21A1 | 81578 | 9746 |
| CPSF2 | 53981 | 8135 |
| DCHS1 | 8642 | 14137 |
| DNAH17 | 8632 | 27298 |
| DNAJC10 | 54431 | 8969 |
| DOCK9 | 23348 | 19186 |
| DYNC1H1 | 1778 | 27420 |
| FAM193A | 8603 | 8372 |
| FLG | 2312 | 13344 |
| GLDC | 2731 | 8759 |
| HMCN1 | 83872 | 39142 |
| HSPBAP1 | 79663 | 3455 |
| IGSF6 | 10261 | 2222 |
| ISG20L2 | 81875 | 2648 |
| ITGA8 | 8516 | 9231 |
| JMJD1C | 221037 | 13810 |
| KDM6A | 7403 | 11359 |
| KIAA0100 | 9703 | 13143 |
| KIAA1671 | 85379 | 12683 |
| KPRP | 448834 | 2921 |
| LYST | 1130 | 23851 |
| MFHAS1 | 9258 | 5852 |
| MLL2 | 8085 | 27398 |
| MYBPC2 | 4606 | 8698 |
| NCKAP1L | 3071 | 9933 |
| NPC1L1 | 29881 | 8897 |
| NPHP3 | 27031 | 1978 |
| NUP188 | 23511 | 13570 |
| ODZ3 | 55714 | 16176 |
| PCLO | 27445 | 27278 |
| PDE4A | 5141 | 8907 |
| PLCG2 | 5336 | 10808 |
| PLXNA2 | 5362 | 17765 |
| PSD4 | 23550 | 8261 |
| RBBP8 | 5932 | 7416 |
| SAMD4A | 23034 | 9419 |
| SCN9A | 6335 | 15069 |
| SMC2 | 10592 | 11297 |
| SNRNP200 | 23020 | 15227 |
| SPAG17 | 200162 | 16454 |
| STAG2 | 10735 | 13183 |
| SYNE1 | 23345 | 57157 |
| TNP2 | 7142 | 995 |
| UGGT1 | 56886 | 19121 |
| UGT1A3 | 54659 | 1066 |
| VCX3B | 425054 | 1764 |
| VPS13D | 55187 | 30041 |
| WDR6 | 11180 | 4895 |
| XIRP2 | 129446 | 15610 |
| XPOT | 11260 | 8684 |
| ZC3H7A | 29066 | 8286 |
| ZFHX4 | 79776 | 16147 |
| ZNF208 | 7757 | 9884 |
| ZNF493 | 284443 | 7280 |

EXAMPLE D demonstrates how additional efforts can be taken to further refine the list of diagnostic genes and tumor-specific signature mutations. For example, EXAMPLE D demonstrates how genomic sequences of coding regions (e.g., exomes) of 53 human bladder cancer tumors can be obtained and analyzed. Of these samples, 45 were the same as those used in EXAMPLES A-C, and 8 were new. Discrepancies between the bladder cancer tumor exomes and a reference human genome can be identified. Such discrepancies can be assessed for reproducibility between sequence reads. The discrepancies that represented real differences between the tumor exome and the reference genome can be classified as variants. These variants can be assessed for novelty by comparing the variants against a database of known single nucleotide polymorphisms. Variants found in the database can be excluded from further analysis. These resulting subsets of variants can be further analyzed for their effect on the encoded protein and mRNA transcript encoding the protein. Variants causing a truncation of the encoded protein through the introduction of stop codons (e.g., nonsense mutations), insertion or deletion mutations that result in a shift in the reading frame (e.g., frameshift mutations), and intronic mutations that potentially alter transcript splicing were categorized as “class 1” variants, having a higher chance in causing a change in function. Variants that resulted in missense mutations, insertions or deletions that maintained the reading frame, and intronic mutations that likely would not alter transcript splicing can be categorized as “class 0” variants, having a lower chance of causing a functional change. Unlike with the previous Examples, variants of both classes (class 1 and class 0) can be included in the gene weighting calculations. Additionally, as described below, the weighting protocol for EXAMPLE D differed from the weighting protocol for the previous Examples.

In addition to the 105 diagnostic genes containing signature mutations revealed through comparison of tumor exomes with a reference human genome, four additional genes containing mutations previously identified as indicators of bladder cancer can be included in the diagnostic methods (See Kompier, L C et al. Bladder cancer: novel molecular characteristics, diagnostics, and therapeutic indications. Urol. Oncol. 2010 January-February; 28(1):91-6 and Huang, F W, et al. Highly recurrent TERT promoter mutations in human melanoma. Science 2013 Feb. 22; 339(6122):957-9). The four additional genes used in EXAMPLE D, for example, are FIBROBLAST GROWTH FACTOR RECEPTOR 3 (FGFR3; (GRCh37): 4:1,795,038-1,810,598; OMIM 134934); PHOSPHATIDYL-INOSITOL 3-KINASE, CATALYTIC, ALPHA (PIK3CA; (GRCh37): 3:178,866,310-178,952,499; OMIM: 171834); V-KI-RAS2 KIRSTEN RAT SARCOMA VIRAL ONCOGENE HOMOLOG (KRAS; (GRCh37): 12:25,358,179-25,403,869; OMIM: 190070); and TELOMERASE REVERSE TRANSCRIPTASE (TERT; (GRCh37): 5:1,253,281-1,295,177; OMIM: 187270). The first three of these additional genes (i.e., FGFR3, PIK3CA, and KRAS) can be treated in the same manner as the diagnostic containing signature mutations revealed through comparison of tumor exomes with a reference human genome.

In addition, all 53 human bladder cancer tumors from EXAMPLE D were screened for the presence or absence of two C to T transitions in the promoter of the TERT gene. The first of these C to T transitions occurs at genomic coordinate Chr5:1,295,228 (GRCh37) and is also referred to as −124G>A or C228T, and the second occurs at genomic coordinate Chr5:1,295,250 (GRCh37) and is also referred to as −146G>A or C250T. (OMIM 187270) Both C228T and C250T generate de novo consensus binding motifs for E-twenty-six (ETS) transcription factors that increase transcriptional activity from the TERT promoter and result in increased expression of telomerase reverse transcriptase, the protein encoded by the TERT gene. (See: Huang, F W, et al. Highly recurrent TERT promoter mutations in human melanoma. Science 2013 Feb. 22; 339(6122):957-9.)

Screening of the TERT gene promoter was done by manual Sanger sequencing. Out of the 53 bladder cancer tumors, 21 were found to carry the mutation C228T, and 3 were found to carry the mutation C250T. The other 29 exome sequences had the wildtype sequence C228/C250. When either of the two TERT promoter mutations (C228T and C250T) were present, they were assigned to variant class 2, since these mutations are known to result in increased transcription of the TERT gene, e.g., they are “activating mutations.”

In general, such activating mutations are included in diagnostic panels because they can be easier to screen (usually with a single amplicon) and they can add to the sensitivity of the diagnostic tests in which they are considered. Further, because the test can be designed to detect a specific, defined variant (e.g. TERT C228T), it can allow for the detection of very rare variants (probably down to 1% of total reads) without the usual concerns about sequencing artifacts.

In EXAMPLE D, variants of class 1 or 0 were both subjected to analyses similar to those conducted in EXAMPLES B & C. In EXAMPLE D, however, the weighting procedure was altered from that described for EXAMPLES B & C. Thus, the weighting protocol can be: Weight (for a given gene)=(((p*l)̂n)/n!)*ê−(p*l), where n=number of unique variants, wherein each unique variant is deweighted by the number of samples in which it appears. In this calculation, each variant (n) is deweighted by the number of samples in which it appears, so that if a variant is unique to 1 sample, it is weighted as 1. If a variant appears in 2 samples, it would be ½, etc. This is in contrast to EXAMPLES B & C, where if a gene had 2 variants, it would have an “n” of 2, even if one variant was unique and the other appears in 10 samples, whereas, in EXAMPLE D that gene's “n” would be 1+( 1/10) or 1.1. As with EXAMPLES B & C, genes identified by comparison of tumor exomes with a reference human genome with a weighted score of less than 10-7 (1×E-7) were retained as diagnostic genes, and the four additional genes FGFR3, PIK3CA, KRAS, and TERT, which were added because mutations in these genes had previously been associated with bladder cancer, were also retained. Altogether, these 109 diagnostic genes are listed in GENE LIST D, and this list of genes, with additional details about the genes, is provided in Table 5. GENE LIST D includes TP53, MLL2, ARID1A, KDM6A, PCLO, C10orf71, ZFHX4, PCNXL2, XIRP2, FOXM1, ODZ3, DNAH17, FLG, PLEC, RP1L1, LOC100130830, OBSCN, NLRP13, AGRN, SPTAN1, PCDHGA2, KPRP, RBBP8, PCDHGA9, OR2T4, AHNAK2, MUC16, RNF111, COL6A1, PCDH8, NACAD, UNC93B1, WDR6, ZRANB3, SRRM2, TMEM175, AKAP13, INPP5D, KIF7, CHD8, NEB, ZSCAN5D, CCDC40, RB1, CAMTA2, KIAA1683, HSPBAP1, GYG2, VPS13D, GLIS2, SUV420H1, JMJD1C, MFHAS1, STAG2, SYNE2, GIMAP6, NUP188, KIF21A, MAGI1, PLXNA2, SCN5A, PLCL2, LIFR, SPEN, KALRN, MAGEC1, LRP1B, C16orf96, SMC2, C7orf58, KNTC1, AZU1, RBM10, PCDHA2, CLCA4, MAST4, ATP2C2, ACTB, INP5F, USH2A, IGSF6, GPR98, NPHP3, ZNF469, CPSF1, TONSL, FAN1, IQSEC2, APOB, RSF1, NBEA, MIR205HG, ZFP36L1, POLE, DST, NVL, ZNFX1, FREM2, PCDHGA2, RECQL5, MLL, HRAS, ERBB2, ERBB3, MLL3, PIK3CA, KRAS, FGFR3, and TERT. The tumor-specific signature mutations that were observed in these 109 diagnostic genes (e.g., MUTATION PANEL D) are disclosed in Example D, and further information about the diagnostic genes is provided in Table 5.

TABLE 5
Diagnostic genes bearing bladder cancer-specific signature mutations

| | | | | | | Samples | |
|---|---|---|---|---|---|---|---|
| | | | | Weighted | Total | Affected | |
| Gene | | Entrez | Gene | Variant | Variant | by all | Weighted |
| # | Gene | Gene ID | Length* | Count** | Count | Variants | Score |
| 1 | TP53 | 7157 | 4858 | 26.333333 | 29 | 25 | 3.85E−62 |
| 2 | MLL2 | 8085 | 27398 | 24 | 24 | 16 | 1.99E−31 |
| 3 | ARID1A | 8289 | 12201 | 13.522222 | 60 | 47 | 2.09E−22 |
| 4 | KDM6A | 7403 | 11359 | 10.5 | 12 | 11 | 8.09E−20 |
| 5 | PCLO | 27445 | 27278 | 12 | 12 | 11 | 6.26E−19 |
| 6 | C10orf71 | 118461 | 5883 | 8 | 8 | 8 | 4.32E−18 |
| 7 | ZFHX4 | 79776 | 16147 | 9.5 | 11 | 11 | 8.77E−18 |
| 8 | PCNXL2 | 80003 | 13423 | 9 | 9 | 9 | 1.99E−17 |
| 9 | XIRP2 | 129446 | 15610 | 9.2 | 14 | 14 | 2.84E−17 |
| 10 | FOXM1 | 2305 | 5403 | 8.0625 | 24 | 20 | 1.41E−15 |
| 11 | ODZ3 | 55714 | 16176 | 10 | 10 | 8 | 3.12E−15 |
| 12 | DNAH17 | 8632 | 27298 | 10 | 10 | 9 | 5.10E−15 |
| 13 | FLG | 2312 | 13344 | 8.333333 | 11 | 10 | 1.35E−14 |
| 14 | PLEC | 5339 | 22434 | 12.077922 | 66 | 43 | 1.98E−14 |
| 15 | RP1L1 | 94137 | 8774 | 8.0625 | 24 | 20 | 3.63E−14 |
| 16 | LOC100130830 | 100130830 | 1934 | 6.946078 | 52 | 38 | 6.10E−14 |
| 17 | OBSCN | 84033 | 45895 | 11.019231 | 63 | 52 | 1.82E−13 |
| 18 | NLRP13 | 126204 | 5346 | 6 | 6 | 6 | 2.10E−13 |
| 19 | AGRN | 375790 | 12458 | 9.185535 | 74 | 53 | 3.40E−13 |
| 20 | SPTAN1 | 6709 | 18304 | 10 | 10 | 7 | 4.79E−13 |
| 21 | PCDHGA2 | 56113 | 2856 | 6 | 6 | 5 | 1.20E−12 |
| 22 | KPRP | 448834 | 2921 | 6 | 6 | 5 | 1.34E−12 |
| 23 | RBBP8 | 5932 | 7416 | 6 | 6 | 6 | 1.48E−12 |
| 24 | PCDHGA9 | 56107 | 2686 | 5 | 5 | 5 | 1.76E−12 |
| 25 | OR2T4 | 127074 | 1246 | 5.527027 | 50 | 39 | 1.94E−12 |
| 26 | AHNAK2 | 113146 | 19572 | 8.03125 | 40 | 35 | 2.42E−12 |
| 27 | MUC16 | 94025 | 60267 | 11.625 | 21 | 16 | 2.81E−12 |
| 28 | RNF111 | 54778 | 8324 | 6 | 6 | 6 | 2.96E−12 |
| 29 | COL6A1 | 1291 | 10407 | 7.026316 | 45 | 39 | 3.63E−12 |
| 30 | PCDH8 | 5100 | 4573 | 6.282258 | 41 | 34 | 3.63E−12 |
| 31 | NACAD | 23148 | 5958 | 6.066667 | 21 | 19 | 4.36E−12 |
| 32 | UNC93B1 | 81622 | 4453 | 6.469231 | 67 | 53 | 4.49E−12 |
| 33 | WDR6 | 11180 | 4895 | 5.333333 | 8 | 8 | 5.45E−12 |
| 34 | ZRANB3 | 84083 | 7877 | 6.5 | 8 | 7 | 6.05E−12 |
| 35 | SRRM2 | 23524 | 11953 | 8 | 8 | 6 | 6.54E−12 |
| 36 | TMEM175 | 84286 | 3949 | 5.361111 | 18 | 17 | 6.72E−12 |
| 37 | AKAP13 | 11214 | 21025 | 8.052632 | 27 | 23 | 7.33E−12 |
| 38 | INPP5D | 3635 | 10174 | 6 | 6 | 6 | 9.78E−12 |
| 39 | KIF7 | 374654 | 8087 | 6.333333 | 9 | 8 | 1.02E−11 |
| 40 | CHD8 | 57680 | 15509 | 6.5 | 8 | 8 | 1.20E−11 |
| 41 | NEB | 4703 | 60665 | 9 | 9 | 9 | 1.27E−11 |
| 42 | ZSCAN5D | 646698 | 2299 | 10.732471 | 143 | 54 | 1.33E−11 |
| 43 | CCDC40 | 55036 | 8034 | 6.667832 | 32 | 26 | 2.06E−11 |
| 44 | RB1 | 5925 | 9918 | 6.5 | 8 | 7 | 2.23E−11 |
| 45 | CAMTA2 | 23125 | 9295 | 6.333333 | 9 | 8 | 2.23E−11 |
| 46 | KIAA1683 | 80726 | 5239 | 6 | 6 | 5 | 2.46E−11 |
| 47 | HSPBAP1 | 79663 | 3455 | 5.071429 | 29 | 27 | 2.47E−11 |
| 48 | GYG2 | 8908 | 5705 | 5.2 | 10 | 10 | 2.54E−11 |
| 49 | VPS13D | 55187 | 30041 | 9 | 9 | 7 | 2.72E−11 |
| 50 | GLIS2 | 84662 | 4662 | 5 | 5 | 5 | 2.75E−11 |
| 51 | SUV420H1 | 51111 | 7894 | 6.018519 | 60 | 54 | 2.90E−11 |
| 52 | JMJD1C | 221037 | 13810 | 7 | 7 | 6 | 2.93E−11 |
| 53 | MFHAS1 | 9258 | 5852 | 6.25 | 31 | 25 | 3.01E−11 |
| 54 | STAG2 | 10735 | 13183 | 7.5 | 9 | 7 | 3.19E−11 |
| 55 | SYNE2 | 23224 | 44698 | 11.632576 | 51 | 32 | 3.55E−11 |
| 56 | GIMAP6 | 474344 | 4244 | 5.076923 | 18 | 17 | 4.51E−11 |
| 57 | NUP188 | 23511 | 13570 | 10.2 | 15 | 8 | 4.84E−11 |
| 58 | KIF21A | 55605 | 13964 | 6 | 6 | 6 | 6.43E−11 |
| 59 | MAGI1 | 9223 | 13986 | 6 | 6 | 6 | 6.49E−11 |
| 60 | PLXNA2 | 5362 | 17765 | 8 | 8 | 6 | 6.92E−11 |
| 61 | SCN5A | 6331 | 14308 | 6 | 6 | 6 | 7.43E−11 |
| 62 | PLCL2 | 23228 | 5760 | 5 | 5 | 5 | 7.89E−11 |
| 63 | LIFR | 3977 | 14497 | 6 | 6 | 6 | 8.03E−11 |
| 64 | SPEN | 23013 | 15178 | 6 | 6 | 6 | 1.05E−10 |
| 65 | KALRN | 8997 | 27441 | 7.5 | 9 | 8 | 1.27E−10 |
| 66 | MAGEC1 | 9947 | 5072 | 7.183824 | 32 | 20 | 1.37E−10 |
| 67 | LRP1B | 53353 | 34487 | 8 | 8 | 7 | 1.37E−10 |
| 68 | C16orf96 | 342346 | 6500 | 5 | 5 | 5 | 1.44E−10 |
| 69 | SMC2 | 10592 | 11297 | 10 | 10 | 5 | 1.44E−10 |
| 70 | C7orf58 | 79974 | 10383 | 6.025 | 46 | 41 | 1.53E−10 |
| 71 | KNTC1 | 9735 | 18824 | 7 | 7 | 6 | 1.85E−10 |
| 72 | AZU1 | 566 | 1907 | 4 | 4 | 4 | 1.93E−10 |
| 73 | RBM10 | 8241 | 6989 | 5 | 5 | 5 | 2.06E−10 |
| 74 | PCDHA2 | 56146 | 2818 | 4.25 | 8 | 8 | 2.08E−10 |
| 75 | CLCA4 | 22802 | 5937 | 6 | 9 | 7 | 2.25E−10 |
| 76 | MAST4 | 375449 | 17298 | 6 | 6 | 6 | 2.29E−10 |
| 77 | ATP2C2 | 9914 | 8224 | 6 | 6 | 5 | 2.32E−10 |
| 78 | ACTB | 60 | 2747 | 6 | 6 | 4 | 2.48E−10 |
| 79 | INPP5F | 22876 | 9298 | 6.043478 | 32 | 27 | 2.71E−10 |
| 80 | USH2A | 7399 | 34444 | 7 | 7 | 7 | 2.83E−10 |
| 81 | IGSF6 | 10261 | 2222 | 4 | 4 | 4 | 3.55E−10 |
| 82 | GPR98 | 84059 | 36956 | 11.085965 | 60 | 34 | 4.03E−10 |
| 83 | NPHP3 | 27031 | 1978 | 4.75 | 10 | 8 | 4.23E−10 |
| 84 | ZNF469 | 84627 | 13485 | 7.11014 | 70 | 52 | 4.42E−10 |
| 85 | CPSF1 | 29894 | 8236 | 5 | 5 | 5 | 4.66E−10 |
| 86 | TONSL | 4796 | 8248 | 5 | 5 | 5 | 4.70E−10 |
| 87 | FAN1 | 330554 | 8276 | 5 | 5 | 5 | 4.78E−10 |
| 88 | IQSEC2 | 23096 | 10549 | 5.25 | 9 | 9 | 4.78E−10 |
| 89 | APOB | 338 | 19621 | 6 | 6 | 6 | 4.83E−10 |
| 90 | RSF1 | 51773 | 8318 | 5 | 5 | 5 | 4.90E−10 |
| 91 | NBEA | 26960 | 22790 | 7 | 7 | 6 | 5.73E−10 |
| 92 | MIR205HG | 642587 | 1441 | 5.136364 | 38 | 26 | 5.74E−10 |
| 93 | ZFP36L1 | 677 | 3420 | 6 | 6 | 4 | 5.95E−10 |
| 94 | POLE | 5426 | 16635 | 6.2 | 11 | 10 | 6.14E−10 |
| 95 | DST | 667 | 38618 | 7 | 7 | 7 | 6.18E−10 |
| 96 | NVL | 4931 | 7367 | 4.833333 | 9 | 9 | 6.31E−10 |
| 97 | ZNFX1 | 57169 | 10057 | 6 | 6 | 5 | 6.31E−10 |
| 98 | FREM2 | 341640 | 20837 | 6 | 6 | 6 | 6.89E−10 |
| 99 | PCDHGA5 | 56110 | 2641 | 4 | 4 | 4 | 7.07E−10 |
| 100 | RECQL5 | 9400 | 9470 | 5.547619 | 28 | 25 | 7.70E−10 |
| 101 | MLL | 4297 | 23722 | 5.5 | 7 | 7 | 1.16E−08 |
| 102 | HRAS | 3265 | 2275 | 5 | 5 | 3 | 5.39E−08 |
| 103 | ERBB2 | 2064 | 10513 | 5 | 5 | 4 | 9.03E−08 |
| 104 | ERBB3 | 2065 | 11108 | 4.5 | 6 | 5 | 4.07E−07 |
| 105 | MLL3 | 58508 | 28340 | 10.65846 | 86 | 31 | 7.45E−07 |
| 107 | PIK3CA | 5290 | 7686 | 3 | 3 | 3 | 5.96E−06 |
| 108 | KRAS | 3845 | 6615 | 2.5 | 4 | 4 | 4.07E−05 |
| 106 | FGFR3 | 2261 | 7474 | 3.238095 | 26 | 23 | 8.22E−06 |
| 109 | TERT | 7015 | NA† | NA† | 2‡ | 24 | NA† |

Combining GENE LIST A, GENE LIST B, GENE LIST C, and GENE LIST D, and removing any duplications, results in a list of 184 diagnostic genes, referred to herein as GENE LIST Z. Specifically, GENE LIST Z includes:

ABCA5, ABCB5, ACTB, AGRN, AHNAK2, AKAP13, ANK3, APOB, ARID1A, ATP2C2, AZU1, BTN2A2, C10orf71, C16orf96, C2orf16, C7orf58, C9orf174, CACNA2D3, CAMTA2, CARD6, CCDC168, CCDC40, CCL20, CDH13, CDRT15L2, CEP95, CHD6, CHD8, CLCA4, COL12A1, COL21A1, COL6A1, CPSF1, CPSF2, CRTAC1, DCHS1, DNAH17, DNAJC10, DNHD1, DOCKS, DST, DYNC1H1, ERBB2, ERBB3, FAM193A, FAM75D1, FAN1, FAT1, FGFR3, FLG, FOXM1, FREM2, GIMAP6, GLDC, GLIS2, GPR98, GYG2, HEATR1, HMCN1, HRAS, HSPBAP1, IGSF6, INPP5D, INPP5F, IQSEC2, ISG20L2, ITGA8, JMJD1C, KALRN, KDM6A, KIAA0100, KIAA1671, KIAA1683, KIF13A, KIF21A, KIF7, KNTC1, KPRP, KRAS, LAPTM4B, LIFR, LOC100130830, LOC100133128, LOC653720, LRP1B, LYST, MAGEC1, MAGI1, MAST4, MFHAS1, MIR205HG, MLL, MLL2, MLL3, MMP8, MUC16, MUC5B, MYBPC2, NACAD, NBEA, NCKAP1L, NEB, NF1, NLRP13, NPC1L1, NPHP3, NUP188, NVL, OBSCN, ODZ3, OR2T4, OR5L2, OR8I2, PCDH8, PCDHA2, PCDHGA2, PCDHGA5, PCDHGA9, PCLO, PCNXL2, PDE4A, PIK3CA, PLCG2, PLCL2, PLD1, PLEC, PLXNA2, POLE, POLR3C, PSD4, RB1, RBI, RBBP8, RBM10, RECQL5, RNF111, RP1L1, RSF1, RYR2, SAMD4A, SCAF11, SCN5A, SCN9A, SLC35G6, SMC2, SNRNP200, SPAG17, SPEN, SPTAN1, SRRM2, STAG2, SUV420H1, SYNE1, SYNE2, TERT, TMEM175, TNN, TNP2, TONSL, TP53, UGGT1, UGT1A3, UNC93B1, USH2A, VCAN, VCX3B, VPS13D, WDR6, WWP1, XIRP2, XPOT, ZC3H7A, ZFHX4, ZFP36L1, ZNF208, ZNF217, ZNF469, ZNF493, ZNF623, ZNF705G, ZNF846, ZNFX1, ZRANB3, and ZSCAN5D, all of which were revealed by one or more of the procedures outlined in EXAMPLES A-D, and all of which are included in Table 6, below.

TABLE 6
Diagnostic genes bearing bladder cancer-specific signature mutations
(GENE LIST Z)

| Gene | | Entrez | Gene |
|---|---|---|---|
| # | Gene | Gene ID | Length |
| 1 | ABCA5 | 23461 | 16948 |
| 2 | ABCB5 | 340273 | 13391 |
| 3 | ACTB | 60 | 2747 |
| 4 | AGRN | 375790 | 12458 |
| 5 | AHNAK2 | 113146 | 19572 |
| 6 | AKAP13 | 11214 | 21025 |
| 7 | ANK3 | 288 | 28629 |
| 8 | APOB | 338 | 19621 |
| 9 | ARID1A | 8289 | 12201 |
| 10 | ATP2C2 | 9914 | 8224 |
| 11 | AZU1 | 566 | 1907 |
| 12 | BTN2A2 | 10385 | 5367 |
| 13 | C10orf71 | 118461 | 5883 |
| 14 | C16orf96 | 342346 | 6500 |
| 15 | C2orf16 | 84226 | 6400 |
| 16 | C7orf58 | 79974 | 10383 |
| 17 | C9orf174 | 100499483 | 354 |
| 18 | CACNA2D3 | 55799 | 11097 |
| 19 | CAMTA2 | 23125 | 9295 |
| 20 | CARD6 | 84674 | 4694 |
| 21 | CCDC168 | 643677 | 22081 |
| 22 | CCDC40 | 55036 | 8034 |
| 23 | CCL20 | 6364 | 1638 |
| 24 | CDH13 | 1012 | 7714 |
| 25 | CDRT15L2 | 256223 | 1386 |
| 26 | CEP95 | 90799 | 6567 |
| 27 | CHD6 | 84181 | 17737 |
| 28 | CHD8 | 57680 | 15509 |
| 29 | CLCA4 | 22802 | 5937 |
| 30 | COL12A1 | 81578 | 24090 |
| 31 | COL21A1 | 81578 | 9746 |
| 32 | COL6A1 | 1291 | 10407 |
| 33 | CPSF1 | 29894 | 8236 |
| 34 | CPSF2 | 53981 | 8135 |
| 35 | CRTAC1 | 55118 | 5948 |
| 36 | DCHS1 | 8642 | 14137 |
| 37 | DNAH17 | 8632 | 27298 |
| 38 | DNAJC10 | 54431 | 8969 |
| 39 | DNHD1 | 144132 | 22844 |
| 40 | DOCK9 | 23348 | 19186 |
| 41 | DST | 667 | 38618 |
| 42 | DYNC1H1 | 1778 | 27420 |
| 43 | ERBB2 | 2064 | 10513 |
| 44 | ERBB3 | 2065 | 11108 |
| 45 | FAM193A | 8603 | 8372 |
| 46 | FAM75D1 | 389763 | 5629 |
| 47 | FAN1 | 330554 | 8276 |
| 48 | FAT1 | 2195 | 19980 |
| 49 | FGFR3 | 2261 | 7474 |
| 50 | FLG | 2312 | 13344 |
| 51 | FOXM1 | 2305 | 5403 |
| 52 | FREM2 | 341640 | 20837 |
| 53 | GIMAP6 | 474344 | 4244 |
| 54 | GLDC | 2731 | 8759 |
| 55 | GLIS2 | 84662 | 4662 |
| 56 | GPR98 | 84059 | 36956 |
| 57 | GYG2 | 8908 | 5705 |
| 58 | HEATR1 | 55127 | 14505 |
| 59 | HMCN1 | 83872 | 39142 |
| 60 | HRAS | 3265 | 2275 |
| 61 | HSPBAP1 | 79663 | 3455 |
| 62 | IGSF6 | 10261 | 2222 |
| 63 | INPP5D | 3635 | 10174 |
| 64 | INPP5F | 22876 | 9298 |
| 65 | IQSEC2 | 23096 | 10549 |
| 66 | ISG20L2 | 81875 | 2648 |
| 67 | ITGA8 | 8516 | 9231 |
| 68 | JMJD1C | 221037 | 13810 |
| 69 | KALRN | 8997 | 27441 |
| 70 | KDM6A | 7403 | 11359 |
| 71 | KIAA0100 | 9703 | 13143 |
| 72 | KIAA1671 | 85379 | 12683 |
| 73 | KIAA1683 | 80726 | 5239 |
| 74 | KIF13A | 63971 | 14303 |
| 75 | KIF21A | 55605 | 13964 |
| 76 | KIF7 | 374654 | 8087 |
| 77 | KNTC1 | 9735 | 18824 |
| 78 | KPRP | 448834 | 2921 |
| 79 | KRAS | 3845 | 6615 |
| 80 | LAPTM4B | 55353 | 3631 |
| 81 | LIFR | 3977 | 14497 |
| 82 | LOC100130830 | 100130830 | 1934 |
| 83 | LOC100133128 | 100133128 | 620 |
| 84 | LOC653720 | 653720 | 5565 |
| 85 | LRP1B | 53353 | 34487 |
| 86 | LYST | 1130 | 23851 |
| 87 | MAGEC1 | 9947 | 5072 |
| 88 | MAGI1 | 9223 | 13986 |
| 89 | MAST4 | 375449 | 17298 |
| 90 | MFHAS1 | 9258 | 5852 |
| 91 | MIR205HG | 642587 | 1441 |
| 92 | MLL | 4297 | 23722 |
| 93 | MLL2 | 8085 | 27398 |
| 94 | MLL3 | 58508 | 28340 |
| 95 | MMP8 | 4317 | 4933 |
| 96 | MUC16 | 94025 | 60267 |
| 97 | MUC5B | 727897 | 26870 |
| 98 | MYBPC2 | 4606 | 8698 |
| 99 | NACAD | 23148 | 5958 |
| 100 | NBEA | 26960 | 22790 |
| 101 | NCKAP1L | 3071 | 9933 |
| 102 | NEB | 4703 | 60665 |
| 103 | NF1 | 4763 | 24550 |
| 104 | NLRP13 | 126204 | 5346 |
| 105 | NPC1L1 | 29881 | 8897 |
| 106 | NPHP3 | 27031 | 1978 |
| 107 | NUP188 | 23511 | 13570 |
| 108 | NVL | 4931 | 7367 |
| 109 | OBSCN | 84033 | 45895 |
| 110 | ODZ3 | 55714 | 16176 |
| 111 | OR2T4 | 127074 | 1246 |
| 112 | OR5L2 | 26338 | 1135 |
| 113 | OR8I2 | 120586 | 1132 |
| 114 | PCDH8 | 5100 | 4573 |
| 115 | PCDHA2 | 56146 | 2818 |
| 116 | PCDHGA2 | 56113 | 2856 |
| 117 | PCDHGA5 | 56110 | 2641 |
| 118 | PCDHGA9 | 56107 | 2686 |
| 119 | PCLO | 27445 | 27278 |
| 120 | PCNXL2 | 80003 | 13423 |
| 121 | PDE4A | 5141 | 8907 |
| 122 | PIK3CA | 5290 | 7686 |
| 123 | PLCG2 | 5336 | 10808 |
| 124 | PLCL2 | 23228 | 5760 |
| 125 | PLD1 | 5337 | 10966 |
| 126 | PLEC | 5339 | 22434 |
| 127 | PLXNA2 | 5362 | 17765 |
| 128 | POLE | 5426 | 16635 |
| 129 | POLR3C | 10623 | 4546 |
| 130 | PSD4 | 23550 | 8261 |
| 131 | RB1 | 5925 | 4772 |
| 132 | RB1 | 5925 | 9918 |
| 133 | RBBP8 | 5932 | 7416 |
| 134 | RBM10 | 8241 | 6989 |
| 135 | RECQL5 | 9400 | 9470 |
| 136 | RNF111 | 54778 | 8324 |
| 137 | RP1L1 | 94137 | 8774 |
| 138 | RSF1 | 51773 | 8318 |
| 139 | RYR2 | 6262 | 37260 |
| 140 | SAMD4A | 23034 | 9419 |
| 141 | SCAF11 | 9169 | 10542 |
| 142 | SCN5A | 6331 | 14308 |
| 143 | SCN9A | 6335 | 15069 |
| 144 | SLC35G6 | 643664 | 1593 |
| 145 | SMC2 | 10592 | 11297 |
| 146 | SNRNP200 | 23020 | 15227 |
| 147 | SPAG17 | 200162 | 16454 |
| 148 | SPEN | 23013 | 15178 |
| 149 | SPTAN1 | 6709 | 18304 |
| 150 | SRRM2 | 23524 | 11953 |
| 151 | STAG2 | 10735 | 13183 |
| 152 | SUV420H1 | 51111 | 7894 |
| 153 | SYNE1 | 23345 | 57157 |
| 154 | SYNE2 | 23224 | 44698 |
| 155 | TERT | 7015 | NA |
| 156 | TMEM175 | 84286 | 3949 |
| 157 | TNN | 63923 | 8789 |
| 158 | TNP2 | 7142 | 995 |
| 159 | TONSL | 4796 | 8248 |
| 160 | TP53 | 7157 | 4858 |
| 161 | UGGT1 | 56886 | 19121 |
| 162 | UGT1A3 | 54659 | 1066 |
| 163 | UNC93B1 | 81622 | 4453 |
| 164 | USH2A | 7399 | 34444 |
| 165 | VCAN | 1462 | 15228 |
| 166 | VCX3B | 425054 | 1764 |
| 167 | VPS13D | 55187 | 30041 |
| 168 | WDR6 | 11180 | 4895 |
| 169 | WWP1 | 11059 | 8695 |
| 170 | XIRP2 | 129446 | 15610 |
| 171 | XPOT | 11260 | 8684 |
| 172 | ZC3H7A | 29066 | 8286 |
| 173 | ZFHX4 | 79776 | 16147 |
| 174 | ZFP36L1 | 677 | 3420 |
| 175 | ZNF208 | 7757 | 9884 |
| 176 | ZNF217 | 7764 | 6630 |
| 177 | ZNF469 | 84627 | 13485 |
| 178 | ZNF493 | 284443 | 7280 |
| 179 | ZNF623 | 9831 | 4558 |
| 180 | ZNF705G | 100131980 | 1920 |
| 181 | ZNF846 | 162993 | 3327 |
| 182 | ZNFX1 | 57169 | 10057 |
| 183 | ZRANB3 | 84083 | 7877 |
| 184 | ZSCAN5D | 646698 | 2299 |

* As noted above, the “gene length” for a particular diagnostic gene includes the length, in basepairs, of the exons of that diagnostic gene, plus 100 base pairs from each (e.g., 5′ and 3′) end of any intervening sequences (e.g., introns) within that diagnostic gene, plus 100 base pairs of the untranslated regions of exons abutting the 5′-most and 3′-most ends of the coding region. This length was used in the gene weighting calculations of the studies to detect the diagnostic genes of the disclosure, as described in detail below.

Thus, the mutations identified in the diagnostic genes in Table 6 by the methods disclosed herein, and potentially other mutations in these same genes that cause truncations of the encoded protein, alterations in the amino acid sequence of the encoded protein, or alterations in the splicing of the encoded transcript, have been identified as biomarkers to be used in detecting bladder cancer, or in monitoring for recurrent bladder cancer. Diagnostic tests based upon these biomarkers are disclosed, as are methods of testing and monitoring, and kits for testing for the presence to bladder cancer, and especially recurrent bladder cancer. These methods, tests, and kits are designed to detect bladder cancer, in some embodiments non-invasively by testing for the presence or level of such biomarkers in the diagnostic genes of the disclosure, for example using DNA isolated from urine samples.

### Diagnostic Genes

Accordingly, in a first aspect, the present disclosure provides methods, systems, and kits for detecting bladder cancer (e.g., diagnosing bladder cancer, monitoring for bladder cancer recurrence, etc.) using 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180 or more genes in any of GENE LISTS A, B, C, D, and/or Z. The diagnostic genes of the disclosure are provided in four different overlapping lists, GENE LISTS A, B, C, and D, and one comprehensive list, GENE LIST Z, and a 61-member subset of that GENE LIST C, GENE LIST X. The 184 individual diagnostic genes of GENE LIST Z are further described in Table 6, above.

The identification of bladder cancer-specific signature mutations in the diagnostic genes of Table 6 enables diagnostic (including monitoring, e.g., recurrence monitoring) and prognostic methods, systems, and kits. The diagnostic genes identified in Table 6, not only provide the particular bladder cancer-specific mutations of MUTATION PANEL A, MUTATION PANEL B, MUTATION PANEL C, and MUTATION PANEL D, but will also harbor additional mutations (e.g., “biomarkers”) that can be used to detect bladder cancer.

### Cancer-Specific Mutations, Biomarkers and Diagnostic Test Panels

In another aspect, the present disclosure provides methods, systems, and kits using particular bladder cancer-specific mutations identified in the diagnostic genes in Table 6—mutations that comprise the bladder cancer-specific signature mutations of the disclosure. These mutations, as identified by the procedures described in EXAMPLES A, B, C and D, are referred to as MUTATION PANEL A, MUTATION PANEL B, MUTATION PANEL C, and MUTATION PANEL D respectively, and are described in detail in Tables A, B, C, and D, respectively. Thus, all of the mutations provided in Tables A, B, C, and D comprise indicators of bladder cancer that can be used in the methods of detecting bladder cancer, as further disclosed herein. The mutations described in Tables A, B, C, and D are collectively referred to as bladder cancer-specific signature mutations of the present disclosure.

In another aspect, the present disclosure provides methods, systems, and kits using indicators of bladder cancer that in this aspect are mutations which result in truncation of the protein encoded by one of the diagnostic genes listed in Tables 1-10, alteration of the amino acid sequence of the encoded protein, or alteration of the splicing of the transcript of one of the diagnostic genes listed in Tables 1-10, but that are not present in MUTATION PANEL A, MUTATION PANEL B, MUTATION PANEL C, or MUTATION PANEL D. Collectively, these additional indicators of bladder cancer, and the bladder cancer-specific signature mutations presented in MUTATION PANEL A, MUTATION PANEL B, MUTATION PANEL C, or MUTATION PANEL D, are biomarkers for bladder cancer in accordance with the present disclosure, and have value in the methods, systems, and kits disclosed below.

Since some bladder cancer-specific signature mutations of the present disclosure may be of greater predictive value than others, or can exhibit a greater frequency of occurrence than others, diagnostic test panels of mutations comprising a predetermined subset of diagnostic genes are envisioned. Such diagnostic test panels can be used for the detection of bladder cancer, or for the monitoring of bladder cancer recurrence, by determining the presence or absence of a limited, and predetermined set of bladder cancer-specific signature mutations of the disclosure.

In some embodiments, such diagnostic test panels of mutations can comprise a single mutation from MUTATION PANEL A, MUTATION PANEL B, MUTATION PANEL C, or MUTATION PANEL D for each one of the 184 diagnostic genes listed in Table 6. In other embodiments, such diagnostic test panels of mutations can comprise the full set of disclosed bladder cancer-specific signature mutations for each single diagnostic gene, but for only a subset of the diagnostic genes listed in Table 6, such as the subset provided in Table 4. In still other embodiments a diagnostic test panel can comprise a subset of the bladder cancer-specific signature mutations as disclosed in MUTATION PANEL A, MUTATION PANEL B, MUTATION PANEL C, or MUTATION PANEL D, for a subset of diagnostic genes listed in Table 6, such as the subset provided in Table 4. In some embodiments, diagnostic methods of the disclosure may comprise determining whether a patient sample has one or more mutations in at least 2, 3, 4, 5, 6, 7, 8, 9, 10, 15, 20, 25, 30, 35, 40, 45, 50 or more of the genes listed in Tables 1-10 or in GENE LIST A, GENE LIST B, GENE LIST C, GENE LIST D, GENE LIST X, or GENE LIST Z. In some embodiments, diagnostic test panels may comprise all cancer-specific signature mutations known for any single diagnostic gene listed in Table 6, with the diagnostic panel in these embodiments comprising 2, 3, 4, 5, 6, 7, 8, 9, 10, 15, 20, 25, 30, 35, 40, 45, 50 or more of the diagnostic genes listed in Tables 1-10 or in GENE LIST A, GENE LIST B, GENE LIST C, GENE LIST D, GENE LIST X, or GENE LIST Z. In other embodiments, diagnostic test panels may comprise a predetermined set of 10, 20, 40, 60, 80, 100, 200, 400, 600, or 800 of the cancer-specific signature mutations disclosed in MUTATION PANEL A, MUTATION PANEL B, and MUTATION PANEL C. In some embodiments, a diagnostic test panel may comprise only those bladder cancer-specific signature mutations with high predictive value that are found most frequently in bladder cancer.

### Diagnostic Methods of the Invention

In another embodiment, the present disclosure also provides methods of detecting bladder cancer (whether for initial diagnosis or monitoring for recurrent bladder cancer) that utilize the disclosed indicators of bladder cancer, including the bladder cancer-specific signature mutations of the disclosure, as identified in the diagnostic genes of the disclosure, to classify patients as having bladder cancer or having recurrent bladder cancer.

Specifically, in one aspect the present disclosure provides an in vitro method of detecting bladder cancer in patients, comprising:

(1) analyzing nucleic acid (e.g., DNA) derived from a biological sample (e.g., urine); to detect the presence or absence of at least one indicator of bladder cancer in said nucleic acid, wherein said indicator is a mutation (e.g., a signature mutation listed in Tables A, B, C, or D, or another missense mutation, nonsense mutation, frameshift mutation, splicing mutation, or large rearrangement, or a combination thereof) in at least 1, 2, 3, 4, 5, 6, 7, 8, 9, 10, 12, 14, 15, 16, 18, 20, 25, 30, 35, 40, 45, 50, 55, 60, 61, 65, 70, 75, 80, 85, 90, 95, 99, 100, 105, 110, 115, 120, 125, 130, 135, 140, 145, 150, 155, 160, 165, 170, 175, 180, or 184 gene(s) listed in Table 6 or Table 4; and

(2) diagnosing a patient in whose sample the presence of said indicator of bladder cancer is detected as having bladder cancer (or as having a high risk of having bladder cancer).

Optionally, this method further includes a step (2)(b), which comprises the optional step of diagnosing a patient in whose sample the absence of any indicator of bladder cancer is detected as not having bladder cancer (or as having a low risk of having bladder cancer).

Optionally, but not necessarily, this method further comprises a step of generating a risk profile for the patient using the results of steps (1) and (2)

In another aspect, the present disclosure provides an in vitro method of monitoring for recurrent bladder cancer in a patient, comprising:

(1) optionally identifying a patient in need of such monitoring;

(2) analyzing nucleic acid (e.g., DNA) derived from a biological sample (e.g., urine) to detect the presence of at least one indicator of bladder cancer in said nucleic acid from said sample, wherein said indicator is a mutation (e.g., a signature mutation listed in Table A, B, C, or D, or another missense mutation, nonsense mutation, frameshift mutation, splicing mutation, or large rearrangement, or a combination thereof) in at least 1, 2, 3, 4, 5, 6, 7, 8, 9, 10, 12, 14, 15, 16, 18, 20, 25, 30, 35, 40, 45, 50, 55, 60, 61, 65, 70, 75, 80, 85, 90, 95, 99, 100, 105, 110, 115, 120, 125, 130, 135, 140, 145, 150, 155, 160, 165, 170, 175, 180, or 184 gene(s) listed in Table 6 or Table 4; and

(3) diagnosing a patient in whose sample the presence of said indicator of bladder cancer is detected as having recurrent bladder cancer (or as having a high risk of having recurrent bladder cancer).

Optionally, this method further includes a step (3)(b), which comprises diagnosing a patient in whose sample the absence of any indicator of bladder cancer is detected as not having recurrent bladder cancer (or as having a low risk of having recurrent bladder cancer).

Optionally, but not necessarily, this method further comprises a step of generating a risk profile for the patient using the results of steps (1), (2), and (3).

For both sets of methods, in some embodiments the determining step may rely exclusively on detecting the presence of one or more of the bladder cancer-specific signature mutations of the disclosure, as disclosed in MUTATION PANEL A, MUTATION PANEL B, MUTATION PANEL C, or MUTATION PANEL D, or some combination thereof. In some embodiments, for both sets of methods, the determining step may comprise detecting the presence of one or more mutations in one or more of the diagnostic genes in Table 6 or Table 4, e.g., mutations that result in a truncation of the encoded protein, and alteration in the amino acid sequence of the encoded protein, or altered splicing of the transcript of a diagnostic gene of the disclosure, wherein the mutation, or mutations are not listed in MUTATION PANEL A, MUTATION PANEL B, MUTATION PANEL C, or MUTATION PANEL D. In some embodiments, for both sets of methods, the determining step may comprise some combination of detecting the presence of one or more of the bladder cancer-specific signature mutations of the disclosure, and detecting the presence of one or more mutations in one or more of the diagnostic genes in Table 6 or Table 4 that are not listed in MUTATION PANEL A, MUTATION PANEL B, MUTATION PANEL C, or MUTATION PANEL D (in some embodiments such other mutation(s) resulting in a truncation of the encoded protein, and alteration in the amino acid sequence of the encoded protein, or altered splicing of the transcript of a diagnostic gene of the disclosure). For both sets of methods, in some embodiments the determining step may comprise detecting the presence of one or more of the bladder cancer-specific signature mutations in a particular diagnostic test panel, as described above. When choosing specific diagnostic genes for inclusion in any embodiment of the disclosure, the individual predictive power of each gene (or mutations therein) may be used to rank them in importance. Such rankings may further be used to weight the various genes in a diagnostic panel of the disclosure. The inventors have determined that the diagnostic genes of the disclosure can be ranked in various ways as shown in Tables 1-10 according to the predictive power of each individual gene.

Thus, in some embodiments of each of the various aspects of the disclosure, the plurality of diagnostic genes analyzed for the presence of an indicator of bladder cancer comprises the top 2, 3, 4, 5, 6, 7, 8, 9, 10, 12, 14, 15, 16, 18, 20, 25, 30, 35, 40, 45, 50, 55, 60, 61, 65, 70, 75, 80, 85, 90, 95, 99, 100, 105, 110, 115, 120, 125, 130, 135, 140, 145, 150, 155, 160, 165, 170, 175, 180, or 184 gene(s) genes listed in any of Tables 1-10. In some embodiments the plurality of diagnostic genes comprises at least some number of genes listed in Table 6 (e.g., at least 3, 4, 5, 6, 7, 8, 9, 10, 15, 20, 25, 30, 35, 40, 45, 50 or more) and this plurality of diagnostic genes comprises at least 1, 2, 3, 4, 5, 6, 7, 8, 9, or 10 of the genes listed in Tables 1-10. In some embodiments the plurality of diagnostic genes comprises at least some number of genes listed in Table 6 (e.g., at least 3, 4, 5, 6, 7, 8, 9, 10, 15, 20, 25, 30, 35, 40, 45, 50 or more) and this plurality of diagnostic genes comprises any one, two, three, four, five, six, seven, eight, nine, or ten or all of gene numbers 1 & 2, 1 to 3, 1 to 4, 1 to 5, 1 to 6, 1 to 7, 1 to 8, 1 to 9, or 1 to 10 of any of Tables 1-10. In some embodiments the plurality of diagnostic genes comprises at least some number of genes listed in Table 6 (e.g., at least 3, 4, 5, 6, 7, 8, 9, 10, 15, 20, 25, 30, 35, 40, 45, 50 or more) and this plurality of diagnostic genes comprises any one, two, three, four, five, six, seven, eight, or nine or all of gene numbers 2 & 3, 2 to 4, 2 to 5, 2 to 6, 2 to 7, 2 to 8, 2 to 9, or 2 to 10 of any of Tables 1-10. In some embodiments the plurality of diagnostic genes comprises at least some number of genes listed in Table 6 (e.g., at least 3, 4, 5, 6, 7, 8, 9, 10, 15, 20, 25, 30, 35, 40, 45, 50 or more) and this plurality of diagnostic genes comprises any one, two, three, four, five, six, seven, or eight or all of gene numbers 3 & 4, 3 to 5, 3 to 6, 3 to 7, 3 to 8, 3 to 9, or 3 to 10 of any of Tables 1-10. In some embodiments the plurality of diagnostic genes comprises at least some number of genes listed in Table 6 (e.g., at least 3, 4, 5, 6, 7, 8, 9, 10, 15, 20, 25, 30, 35, 40, 45, 50 or more) and this plurality of diagnostic genes comprises any one, two, three, four, five, six, or seven or all of gene numbers 4 & 5, 4 to 6, 4 to 7, 4 to 8, 4 to 9, or 4 to 10 of any of Tables 1-10. In some embodiments the plurality of diagnostic genes comprises at least some number of genes listed in Table 6 (e.g., at least 3, 4, 5, 6, 7, 8, 9, 10, 15, 20, 25, 30, 35, 40, 45, 50 or more) and this plurality of diagnostic genes comprises any one, two, three, four, five, six, seven, eight, nine, 10, 11, 12, 13, 14, or 15 or all of gene numbers 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Tables 1-10.

### Biological Samples

Biological samples can be obtained from a subject (e.g., a human patient) using any known device or method so long as the analytes to be measured by the methods of detecting are not rendered undetectable by the detection step. Non-limiting examples of devices or methods suitable for taking bodily fluid from a mammal include urine sample cups, urethral catheters, swabs, hypodermic needles, thin needle biopsies, hollow needle biopsies, punch biopsies, metabolic cages, and aspiration.

In order to adjust the expected concentrations of sample analytes in the sample to fall within the expected range of detecting assay, the test sample may be diluted to reduce the concentration of the sample analytes prior to analysis. The degree of dilution may depend on a variety of factors including but not limited to the type of assay used to measure the analytes, the reagents utilized in the assay, and the type of bodily fluid contained in the test sample. Non-limiting examples of suitable diluents include deionized water, distilled water, saline solution, Ringer's solution, phosphate buffered saline solution, TRIS-buffered saline solution, standard saline citrate, and HEPES-buffered saline.

In an embodiment, the biological sample is an amount of bodily fluid obtained from a subject, such as a mammal. Bodily fluids can include urine, blood, plasma, serum, semen, perspiration, tears, mucus, and tissue lystates. In an exemplary embodiment, the sample is urine.

In some embodiments nucleic acid (e.g., DNA, RNA) is obtained from a biological sample (e.g., urine sample) and used to determine the presence (or absence) of an indicator of bladder cancer, which can be DNA from cells (e.g., nucleated cells), cell fragments or microvesicles present in the urine, or cell-free DNA. If the DNA that is obtained from a urine sample and used to determine the presence of an indicator of bladder cancer is from cells present in the urine, the cells may be isolated by sedimentation (e.g., through centrifugation), subsequently lysed, and the DNA extracted from the lysate. If the DNA that is obtained from a urine sample and used to determine the presence of an indicator of bladder cancer is cell-free DNA, it may be isolated by, e.g., ultrafiltration of the urine, or passage of the urine through a cationic matrix that binds the negatively-charged DNA.

### Detecting

For both sets of methods, determining the presence of indicators of bladder cancer, e.g., mutations, can be accomplished by adapting suitable techniques used in the art, of which there are many with which those skilled in the art would be familiar, to the methods disclosed herein. Determining generally refers to the detection of the presence (or absence or level or structure) of a target nucleic acid molecule, which can include a target nucleic acid molecule having a polymorphism or indicator of bladder cancer of interest. A nucleic acid molecule is “detected” as used herein where the level of nucleic acid measured (e.g., by quantitative PCR), or the level of detectable signal provided by the detectable label (including the level of nucleic acid having a certain structure or sequence, e.g., an indicator of bladder cancer) is at all above the background level, thus allowing for a qualitative (e.g., present or absent) or quantative (e.g., amount) detection of the analyte. For example, detection can occur by a process wherein the signal generated by a directly or indirectly labeled probe nucleic acid molecule (capable of hybridizing to a target in a sample) is measure or observed. Detection of the probe nucleic acid is directly indicative of the presence, and thus the detection of, an indicator of bladder cancer, such as a sequence encoding a marker gene.

A target nucleic acid molecule, e.g., an indicator of bladder cancer, can be detected by amplifying a nucleic acid sample in or obtained from a sample obtained from a patient, using, e.g., oligonucleotide primers that are specifically designed to hybridize with a portion of the target nucleic acid sequence. For example, the detecting step may involve detection by a technique chosen from allele-specific polymerase chain reactions (PCR), mutant-enriched PCR, digital protein truncation tests, DNA or RNA sequencing (including, e.g., direct sequencing, massively parallel sequencing), use of molecular beacon probes or primers, BEAMing digital PCR, or allele-specific hybridization. Quantitative amplification methods such as, but not limited to, TaqMan® (a commercially available quantitative PCR system) can also be used to “detect” an indicator of bladder cancer according to the disclosure. Methods and techniques for “detecting” fluorescent, radioactive, and other chemical labels may be found in Ausubel et al. (1995, Short protocols in Molecular biology, 3^{rd} Ed. John Wiley and Sons, Inc.).

Alternatively, a nucleic acid can be “indirectly detected” wherein a moiety is attached to a probe nucleic acid that will hybridize with the target, wherein the moiety comprises, for example, an enzyme activity, allowing detection of the target in the presence of an appropriate substrate, or a specific antigen or other marker allowing detection by addition of an antibody or other specific indicator.

Nucleic acid molecules having for example, indicators of bladder cancer, can be detected and/or isolated by specific hybridization under particular stringency conditions. “Stringency conditions” for hybridization is a term of art that refers to incubation and wash conditions, e.g., conditions of temperature and buffer concentration, which permit hybridization of a particular nucleic acid to a second nucleic acid. The first nucleic acid can be perfectly complementary to the second, or the first and second can share some degree of complementarity less than perfect (e.g., 70%, 75%, 85%, 95%). For example, certain high stringency conditions can be used that distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). The conditions that determine the stringency of hybridization depend on parameters such as, for example, ionic strength (e.g. 0.2×SSC, 0.1×SSC), temperature (e.g., room temperature, 42° C., 68° C.), the concentration of destabilizing agents such as formamide or denaturing agents such as SDS, and factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules.

The detection of bladder cancer, or monitoring for recurrent bladder cancer, can be accomplished with a sensitivity of at least 85% at a specificity of at least 85%, with a sensitivity of at least 80% at a specificity of at least 90%, with a sensitivity of at least 75% at a specificity of at least 95%, or with a sensitivity of at least 70% at a specificity of at least 95%.

### Classifying

The present disclosure provides methods that classify or diagnosing a subject (e.g., a human patient) as having bladder cancer or having recurrent bladder cancer. As used herein, “classifying a cancer” and “cancer classification” refer to determining one or more clinically-relevant features of a cancer and/or determining a particular prognosis of a patient having said cancer. Thus “classifying a cancer” includes, but is not limited to: (i) diagnosis of bladder cancer; (ii) evaluating risk or likelihood of recurrence; (iii) evaluating metastatic potential, potential to metastasize to specific organs, risk of recurrence, and/or course of the tumor; (iv) evaluating tumor stage; (v) determining patient prognosis in the absence of treatment of the cancer; (vi) determining prognosis of patient response (e.g., tumor shrinkage or progression-free survival) to treatment (e.g., surgery to excise tumor, adjuvant therapy, including immunotherapy, targeted therapy, or conventional chemotherapy, etc.); (vii) diagnosis of actual patient response to current and/or past treatment; (viiii) determining a preferred course of treatment for the patient; (ix) prognosis for patient relapse after treatment (either treatment in general or some particular treatment); (x) prognosis of patient life expectancy (e.g., prognosis for overall survival), etc. In some embodiments a recurrence-associated or metastatic progression-associated clinical parameter or increased expression of an indicator indicate a negative classification in cancer (e.g., increased likelihood of recurrence or progression).

In an embodiment a risk profile in generated. The term “risk profile” generally means the likelihood of a subject as described herein having or developing bladder cancer. This involves correlating a particular assay or analysis result or output to some likelihood (e.g., increased, not increased, decreased, etc.) of some clinical feature (e.g., increased risk (e.g., increased hereditary risk) of cancer), or additionally or alternatively concluding or communicating such clinical feature based at least in part on such particular assay or analysis result, such correlating, concluding or communicating may comprise assigning a risk or likelihood of the clinical feature occurring based at least in part on the particular assay or analysis result. In some embodiments, such risk is a percentage probability of the event or outcome occurring. In some embodiments, the patient is assigned to a risk group (e.g., low risk, intermediate risk, high risk, etc.). In some embodiments “low risk” is any percentage probability below 5%, 10%, 15%, 20%, 25%, 30%, 35%, 40%, 45%, or 50%. In some embodiments “intermediate risk” is any percentage probability above 5%, 10%, 15%, 20%, 25%, 30%, 35%, 40%, 45%, or 50% and below 15%, 20%, 25%, 30%, 35%, 40%, 45%, 50%, 55%, 60%, 65%, 70%, or 75%. In some embodiments “high risk” is any percentage probability above 25%, 30%, 35%, 40%, 45%, 50%, 55%, 60%, 65%, 70%, 75%, 80%, 85%, 90%, 95%, or 99%.

The terms “increased,” “normal,” and “decreased” when referring to risk preferably mean that the subject has that risk level when compared with the risk associated with an individual having a different nucleic acid in one or more genes from Tables 1-10, and in GENE LIST A, GENE LIST B, GENE LIST C, GENE LIST D, GENE LIST X, and GENE LIST Z.

Combination with Other Diagnostic Procedures

In another aspect, the results of the methods of the present disclosure can be combined with, or interpreted in light of, the results from other diagnostic procedures involving other bladder cancer biomarkers. Descriptions of other bladder cancer biomarkers and their use have been provided in a recent review article by Parker and Spiess. See, Parker, J. & Spiess, P. E.; TheScientificWorldJournal 11:1103-1112 (2011). Among the other diagnostic procedures that can be combined with methods of the present disclosure for the detection of bladder cancer, or recurrent bladder cancer, are fluorescence in situ hybridization (FISH) to detect aneuploidy (of chromosomes 3, 7 & 17) and loss of heterozygosity (of the 9p21 locus in malignant urothelial cells); expression levels of the nuclear matrix protein 22 (NMP22); immunodetection of the bladder tumor-associated antigen, “BTA,” and bladder carcinoma specific antigens M344, LDQ10, and 19A211; immunodetection of the Lewis X antigen and urinary fibrin/fibrinogen degradation products; determination of telomerase activity, and the presence of hyaluronic acid and hyaluronidase activity; microsatellite analysis for assessing loss of heterozygosity; immunoassays to test for the presence and level of the nuclear matrix protein specific to bladder cancer tissues, BLCA-4; and assessment of levels of expression of the proteins cytokeratin CK20, soluble FAS, and survivin. Ibid. The results of the methods of the present disclosure can also be combined with, or interpreted in light of, the results from immunoflurometric assays designed to test the levels of expression of proteins of the minichromosome maintenance (Mcm) family, and specifically Mcm5, as described by Kelly et al. (PLoS ONE 7(7)e40305:1-8 (2012). The results of the methods of the present disclosure can also be combined with, or interpreted in light of, the assays designed to detect epigenetic changes in the FOXE1, GATA4, TWIST1, NID2, CCNA1 genes, as described in U.S. Patent Application Publication No. 2012/0027870. The results of the methods of the present disclosure can also be combined with, or interpreted in light of, the multiparametric assays described in U.S. Patent Application Publication No. 2012/0244536.

### Methods of Treatment

The present disclosure provides methods that include treatment based on the classification of bladder cancer as described herein. Chemotherapy and surgery are non-limiting examples of treatment options. Those skilled in the art can readily adapt various existing are familiar with various aggressive and less aggressive treatments for bladder cancer for use in the treatment methods described herein. “Active treatment” in bladder cancer is well-understood by those skilled in the art and, as used herein, has the conventional meaning in the art. Generally speaking, active treatment in bladder cancer can include anything other than “watchful waiting.” Active treatment currently applied in the art of bladder cancer treatment can include, e.g., radiotherapy, transurethral resection, cystectomy, hormonal therapy, chemotherapy, immunotherapy, etc. Active treatment can include a drug regimen, which can include, but is not limited to, Adriamycin, Cisplatin, Doxorubicin Hydrochloride, and Platinol. Each treatment option has with it certain risks as well as side-effects of varying severity.

“Watchful-waiting,” also sometimes called “active surveillance,” also has its conventional meaning in the art. This generally means observation and regular monitoring without treatment of the underlying disease. Watching-waiting can also be suggested when the risks of surgery, radiation therapy, hormonal therapy, or chemotherapy, for example, outweighs the possible benefits. Other treatments can be started if symptoms develop, or if there are signs that the cancer growth is accelerating.

In one aspect, the present disclosure provides methods of treating a subject (e.g., a human cancer patient) that includes an in vitro method generally comprising detecting an indicator of bladder cancer in a patient sample; diagnosing a patient in whose sample an indicator of bladder cancer is detected as having bladder cancer; and recommending, prescribing, or administering a treatment for a patient diagnosed as having bladder cancer. For example, the disclosure provides a method of treating bladder cancer patients comprising:
(1) analyzing nucleic acid (e.g., DNA) in or obtained from a biological patient sample (e.g., urine) to detect the presence or absence of at least one indicator of bladder cancer in said nucleic acid, wherein said indicator is a mutation (e.g., a signature mutation listed in Table A, B, C, or D, or another missense mutation, nonsense mutation, frameshift mutation, splicing mutation, or large rearrangement, or combination thereof) in one or more genes listed in Table 6 or Table 4;
(2) diagnosing a patient in whose sample an indicator of bladder cancer is detected in step (1) as having bladder cancer; and
(3) recommending, prescribing, or administering a treatment regimen a treatment for a patient diagnosed as having bladder cancer in step (2).

In one aspect, the present disclosure provides methods of treating a subject (e.g., a human cancer patient) that includes an in vitro method generally comprising detecting bladder cancer in a patient according to the present disclosure, classifying the patient as having bladder cancer, and recommending, prescribing, or administering a treatment for the cancer patient based on the classification. For example, the disclosure provides a method of treating a cancer patient comprising:
(1) analyzing nucleic acid (e.g., DNA) in or obtained from a biological patient sample (e.g., urine) to detect the presence or absence of at least one indicator of bladder cancer in said nucleic acid, wherein said indicator is a mutation (e.g., a signature mutation listed in Table A, B, C, or D, or another missense mutation, nonsense mutation, frameshift mutation, splicing mutation, or large rearrangement, or combination thereof) in one or more genes listed in Table 6 or Table 4;
(2)(a) diagnosing a patient in whose sample the presence of said indicator of bladder cancer is detected as having bladder cancer; or
(2)(b) diagnosing a patient in whose sample the presence of said indicator of bladder cancer is detected as not having bladder cancer; and
(3)(a) recommending, prescribing, or administering an aggressive treatment regimen for a patient diagnosed as having bladder cancer in step (2)(a); or
(3)(b) recommending, prescribing, or administering a passive treatment regimen (e.g., watchful waiting or active surveillance) for a patient diagnosed as not having bladder cancer in step (3)(a).

In one aspect, the present disclosure provides methods of treating a subject (e.g., a human cancer patient) that includes an in vitro method of monitoring for recurrent bladder cancer in a patient, classifying the patient as having recurrent bladder cancer, and recommending, prescribing, or administering a treatment for the cancer patient based on the classification. For example, the disclosure provides a method of treating bladder cancer patients comprising:
(1) optionally identifying a patient in need of monitoring for recurrent bladder cancer;
(2) analyzing nucleic acid (e.g., DNA) in or obtained from a biological sample (e.g., urine) to detect the presence or absence of at least one indicator of bladder cancer in said nucleic acid, wherein said indicator is a mutation (e.g., a signature mutation listed in Table A, B, C, or D, or another missense mutation, nonsense mutation, frameshift mutation, splicing mutation, or large rearrangement, or combination thereof) in one or more genes listed in Table 6 or Table 4;
(3)(a) diagnosing a patient in whose sample the presence of said indicator of bladder cancer is detected as having recurrent bladder cancer; or
(3)(b) diagnosing a patient in whose sample the presence of said indicator of bladder cancer is detected as not having recurrent bladder cancer; and
(4)(a) recommending, prescribing, or administering an aggressive treatment regimen for a patient diagnosed as having recurrent bladder cancer in step (3)(a); or
(4)(b) recommending, prescribing, or administering a passive treatment regimen (e.g., watchful waiting or active surveillance) for a patient diagnosed as not having recurrent bladder cancer in step (3)(b).

In one aspect, the present disclosure provides methods of treating a subject (e.g., a human cancer patient) that includes an in vitro method of monitoring for recurrent bladder cancer in a patient, classifying the patient as having recurrent bladder cancer, and recommending, prescribing, or administering a treatment for the cancer patient based on the classification. For example, the disclosure provides a method of treating a cancer patient comprising:
(1) optionally identifying a patient in need of monitoring for recurrent bladder cancer;
(2) analyzing nucleic acid (e.g., DNA) in or obtained from a biological sample (e.g., urine) from said patient to detect the presence or absence of at least one indicator of bladder cancer in said nucleic acid, wherein said indicator is a mutation (e.g., a signature mutation listed in Table A, B, C, or D, or another missense mutation, nonsense mutation, frameshift mutation, splicing mutation, or large rearrangement, or combination thereof) in one or more genes listed in Table 6 or Table 4;
(3) diagnosing said patient as having recurrent bladder cancer, based at least in part on the presence of said indictor of bladder cancer; and
(4) based at least in part on the classification in step (3), recommending, prescribing, or administering a treatment regimen.

### Compositions

In one aspect, the disclosure provides compositions for use in the above methods. Such compositions include, but are not limited to, nucleic acid probes hybridizing to a set of bladder cancer diagnostic genes (or to any nucleic acids encoded thereby or complementary thereto); nucleic acid primers and primer pairs suitable for amplifying (e.g., by PCR) all or a portion of a set of bladder cancer diagnostic genes or any nucleic acids encoded thereby; antibodies binding immunologically to a polypeptide encoded by a set of bladder cancer diagnostic genes; probe sets comprising a plurality of said nucleic acid probes, nucleic acid primers, antibodies, and/or polypeptides; microarrays comprising any of these; etc. In some aspects, as described herein, the disclosure provides computer methods, systems, software and/or modules for use in the above methods.

In some embodiments the disclosure provides a set of probes comprising isolated (or synthetic) oligonucleotides capable of selectively hybridizing to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more genes from Tables 1-10, and in GENE LIST A, GENE LIST B, GENE LIST C, GENE LIST D, GENE LIST X, and GENE LIST Z. The terms “probe” and “oligonucleotide” (also “oligo”), when used in the context of nucleic acids, interchangeably refer to a relatively short nucleic acid fragment. The disclosure also provides primers useful in the methods of the disclosure. “Primers” are oligonucleotides capable, under the right conditions and with the right companion reagents, of selectively priming the biochemical synthesis of (e.g., amplifying) a target nucleic acid (e.g., a target gene or portion thereof). In the context of nucleic acids, “probe” is used herein to encompass “primer” since primers can generally also serve as probes.

The probe can generally be of any suitable size/length. In some embodiments the probe has a length from about 8 to 200, 15 to 150, 15 to 100, 15 to 75, 15 to 60, or 20 to 55 bases in length. They can be labeled with detectable markers with any suitable detection marker including but not limited to, radioactive isotopes, fluorophores, biotin, enzymes (e.g., alkaline phosphatase), enzyme substrates, ligands and antibodies, etc. See Jablonski et al., Nucleic Acids Research 14:6115-6128 (1986); Nguyen et al., Biotechniques 13:116-123 (1992); Rigby et al., J. Mol. Bio. 113:237-251 (1977). Indeed, probes may be modified in any suitable manner for various molecular biological applications. General techniques for producing such oligonucleotide probes are conventional in the art and, based on the present disclosure, can be adapted and applied to the present disclosure to produce compositions of the disclosure.

Probes according to the disclosure can be used in the hybridization/amplification/detection techniques discussed above. Thus, some embodiments of the disclosure comprise probe sets suitable for use in a microarray in detecting, amplifying and/or quantitating a plurality of bladder cancer diagnostic genes. In some embodiments the probe sets have a certain proportion of their probes directed to bladder cancer diagnostic genes—e.g., a probe set comprising at least 10%, 20%, 30%, 40%, 50%, 55%, 60%, 65%, 70%, 75%, 80%, 85%, 90%, 95%, 96%, 97%, 98%, 99%, or 100% probes specific for bladder cancer diagnostic genes according to the present disclosure. In some embodiments the probe set comprises probes directed to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more genes from Tables 1-10, and in GENE LIST A, GENE LIST B, GENE LIST C, GENE LIST D, GENE LIST X, and GENE LIST Z. Such probe sets can be incorporated into high-density arrays comprising 5,000, 10,000, 20,000, 50,000, 100,000, 200,000, 300,000, 400,000, 500,000, 600,000, 700,000, 800,000, 900,000, or 1,000,000 or more different probes. In other embodiments the probe sets comprise primers (e.g., primer pairs) for amplifying nucleic acids comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more genes from Tables 1-10, and in GENE LIST A, GENE LIST B, GENE LIST C, GENE LIST D, GENE LIST X, and GENE LIST Z.

In one aspect the present disclosure provides the use of such compositions. In one embodiment, for example, the disclosure provides the use of a plurality of oligonucleotide probes for detecting bladder cancer in a patient sample, wherein said plurality of probes comprises at least one probe selectively hybridizing to each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more genes from Tables 1-10, and in GENE LIST A, GENE LIST B, GENE LIST C, GENE LIST D, GENE LIST X, and GENE LIST Z. The disclosure also provides the use of an oligonucleotide probe set for detecting bladder cancer in a patient sample, wherein said probe set comprises at least one probe per gene directed to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more genes from Tables 1-10, and in GENE LIST A, GENE LIST B, GENE LIST C, GENE LIST D, GENE LIST X, and GENE LIST Z. The disclosure also provides the use of a plurality of oligonucleotide primers for detecting bladder cancer in a patient sample, wherein said plurality of primers comprises at least one primer selectively hybridizing to each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more genes from Tables 1-10, and in GENE LIST A, GENE LIST B, GENE LIST C, GENE LIST D, GENE LIST X, and GENE LIST Z. In other embodiments the probe sets comprise primers (e.g., primer pairs) for amplifying nucleic acids comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more genes from Tables 1-10, and in GENE LIST A, GENE LIST B, GENE LIST C, GENE LIST D, GENE LIST X, and GENE LIST Z.

### Kits

In another aspect, the present disclosure provides kits for conducting the methods of the present disclosure that utilize the disclosed indicators of bladder cancer, including, but not limited to, the bladder cancer-specific signature mutations of the disclosure, as identified in the diagnostic genes of the disclosure, to classify patients as having bladder cancer or having recurrent bladder cancer.

Such kits may comprise reagents useful, sufficient, or necessary for detecting and/or characterizing at least one indicator of bladder cancer in nucleic acid (e.g., DNA) from a biological sample (e.g., urine sample) from a patient (e.g., a human patient), said at least one indicator being chosen from one or more mutation (e.g., a signature mutation listed in Table A, B, C, or D, or another missense mutation, nonsense mutation, frameshift mutation, a splicing mutation, or large rearrangement, or a combination thereof) in a gene listed in Table 6. Such kits may also comprise instructions for using the kit, and preferably instructions on using the kit to practice a diagnostic method of the present disclosure using samples (e.g., human samples), in some embodiments urine samples.

Such kits may optionally comprise additional reagents and devices required for obtaining nucleic acid (e.g., DNA) from a biological (e.g., urine) sample, including centrifuge tubes for isolating cells from a sample, filtration or ultrafiltration devices for isolating cells or cell-free nucleic acid from a sample, reagents for lysing cells isolated from a sample, reagents for extracting nucleic acid from cell lysates, cationic matrices or media, including matrices and media installed in cationic spin-tubes or spin-columns, for binding cell-free nucleic acid or binding nucleic acid extracted from cell lysates, and any reagent necessary for elution of bound nucleic acid from such cationic matrices or media, and storage of such eluted nucleic acid.

Such kits may also optionally comprise reagents in dry or liquid form that are intended to be added directly to samples (e.g., urine samples) to inhibit degradation of nucleic acid (e.g., DNA or RNA), including such reagents as ethylenediaminetetraacetic acid (EDTA) or other preservatives, protease inhibitors such as proteinase K, nuclease inhibitors, antimicrobial agents such as sodium azide, buffers, salts, etc.

The reagents useful, sufficient, or necessary for detecting and/or characterizing at least one indicator of bladder cancer in nucleic acid (e.g., DNA) from a biological (e.g., urine) sample can comprise any reagent know in the art for use in any technique designed to detection mutations and alterations in nucleic acids. For example, the reagents included can be reagents useful for specific detection of mutations by allele-specific polymerase chain reactions (PCR), mutant-enriched PCR, digital protein truncation tests, DNA or RNA sequencing (e.g., direct sequencing, massively parallel sequencing), use of molecular beacon probes or primers, BEAMing digital PCR, or allele-specific hybridization.

Hence, among the reagents that can be included are oligonucleotide primers suitable for the amplification of a target nucleic acid sequence, DNA polymerases such as thermostable DNA polymerases to be used in amplification reactions and other types of nucleic acid template-directed synthetic reactions, other nucleic acid modifying enzymes such as RNase A and RNase H, nucleotides (including deoxyribonucleotides and modified nucleotides, such as fluorescently-labeled dideoxynucleotides for terminating amplification reactions), buffers, and other additives required for amplification of target sequences from isolated nucleic acid, and for sequencing such amplified nucleic acid, or isolated nucleic acid. Other reagents that can be included are probes, such as fluorescently-labeled probes, designed to detect specific nucleotide sequences in isolated and/or amplified nucleic acid. Also included can be so-called “gene chips” that comprise a microarray of oligonucleotides that can have a nucleotide sequence containing one of the signature mutations listed in the accompanying tables in context with surrounding genomic nucleotide sequence, or the complement thereof.

Also included in the kits of the disclosure can be reagents necessary and sufficient to serve as positive and negative controls for the method being used to detect the signature mutations of the present disclosure.

Regardless of the technique or method being used to detect at least one indicator of bladder cancer in nucleic acid (e.g., DNA) from a biological (e.g., urine) sample from a patient (e.g., a human patient), including at least one signature mutation of the present disclosure, the kit may be designed to include only those reagents useful, sufficient, or necessary for detecting and/or characterizing a subset of the signature mutations listed in Table A, B, C, or D, such as, for example only those mutations that comprise a particular diagnostic test panel. Consequently, different types of kits, each type designed to test for mutants that comprise a particular diagnostic test panel, are envisioned. Additionally, different types of kits, each type designed to test for mutants that comprise a particular diagnostic test panel using a particular mutation screening method, are envisioned.

Finally, kits may designed to only detect signature mutations in a particular diagnostic gene, or a particular set of diagnostic genes, selected from all diagnostic genes identified in Table 6. For example, kits may be designed to detect indicators of bladder cancer in (e.g., the coding sequence of) only those diagnostic genes in GENE LIST X, as identified in Table 4.

The following examples will serve to illustrate various aspects and/or features of the disclosure and are not to be regarded as limitations of the scope of the disclosure.

### Automated Method for Diagnosing, Monitoring, or Determining Bladder Cancer

In an embodiment, a system for diagnosing, monitoring, or determining bladder cancer in a subject (e.g., a human patient) is provided that includes a database to store a plurality of bladder cancer database entries, and a processing device that includes the modules of a bladder cancer determining application. In this embodiment, the modules are executable by the processing device, and include an analyte input module, a comparison module, and an analysis module.

The analyte input module receives three or more sample analyte concentrations that include the biomarker analytes. In one embodiment, the sample analyte concentrations are entered as input by a user of the application. In another embodiment, the sample analyte concentrations are transmitted directly to the analyte input module by the sensor device used to measure the sample analyte concentration via a data cable, infrared signal, wireless connection or other methods of data transmission known in the art.

The comparison module compares each sample analyte concentration to an entry of a bladder cancer database. Each entry of the bladder cancer database includes a list of minimum diagnostic concentrations reflective of a bladder cancer. The entries of the bladder cancer database may further contain additional minimum diagnostic concentrations to further define diagnostic criteria including but not limited to minimum diagnostic concentrations for additional types of bodily fluids, additional types of subjects, and severities of a particular cancer.

The analysis module determines a bladder cancer by combining the bladder cancers identified by the comparison module for all of the sample analyte concentrations. In one embodiment, the bladder cancer has the most minimum diagnostic concentration that is less than the corresponding sample analyte concentrations. In another embodiment, the bladder cancer has the most minimum diagnostic concentrations that are all less than the corresponding sample analyte concentrations. In yet other embodiments, the analysis module combines the sample analyte concentrations algebraically to calculate a combined sample analyte concentration that is compared to a combined minimum diagnostic concentration calculated from the corresponding minimum diagnostic criteria using the same algebraic operations. Other combinations of sample analyte concentrations from within the same test sample, or combinations of sample analyte concentrations from two or more different test samples containing two or more different bodily fluids may be used to determine a bladder cancer.

The system includes one or more processors and volatile and/or nonvolatile memory and can be embodied by or in one or more distributed or integrated components or systems. The system may include computer readable media (CRM) on which one or more algorithms, software, modules, data, and/or firmware is loaded and/or operates and/or which operates on the one or more processors to implement the systems and methods identified herein. The computer readable media may include volatile media, nonvolatile media, removable media, non-removable media, and/or other media or mediums that can be accessed by a general purpose or special purpose computing device. For example, computer readable media may include computer storage media and communication media, including but not limited to computer readable media. Computer storage media further may include volatile, nonvolatile, removable, and/or non-removable media implemented in a method or technology for storage of information, such as computer readable instructions, data structures, program modules, and/or other data. Communication media may, for example, embody computer readable instructions, data structures, program modules, algorithms, and/or other data, including but not limited to as or in a modulated data signal. The communication media may be embodied in a carrier wave or other transport mechanism and may include an information delivery method. The communication media may include wired and wireless connections and technologies and may be used to transmit and/or receive wired or wireless communications. Combinations and/or sub-combinations of the above and systems, components, modules, and methods and processes described herein may be made.

### EXAMPLES

### Example A

### Identification of a First Panel of Gene Mutations Associated with Bladder Cancer (MUTATION PANEL A)

48 fresh-frozen human bladder tumor samples were processed through the Illumina Tru-seq Capture Protocol (Illumina, Inc.; San Diego, Calif.), and isolated tumor genomic DNA was sequenced using a massively parallel sequencing procedure employing an Illumina HiSeq2000 Sequencing Apparatus (Illumina, Inc.; San Diego, Calif.). The resulting reads were aligned against HG19, and all discrepancies were cataloged. Discrepancies that occurred in 20% or greater of the reads were classified as variants. The variant calls were then compared with the dbSNP database; any variants that were present within dbSNP were excluded from further analysis. Variants were then classified in 3 categories: synonymous, missense, and suspected deleterious. Synonymous variants have nucleic but no amino acid changes, and were ignored. Missense variant result in single amino acid changes, and may be detrimental to gene function. Suspected deleterious variants are stops (e.g., nonsense mutations), or insertions or deletions that result in a frameshift; these both lead to truncations of the gene. Genes having at least one frameshift or nonsense mutation were considered for further analysis. The individual variants considered during further analysis of the candidate diagnostic genes are shown in Table 7 as belonging to “Variant Class 1,” which, in this study, comprised stop mutations, frameshift mutations and missense mutations. Variants excluded from these analyses are shown in Table 7 as belonging to “Variant Class 0,” which comprised synonymous mutations and splice site mutations. Genes were then weighted based on their missense/suspected deleterious variants with the following formula:

Weight(for a given gene)=((# of Unique Variantŝ2)/(# of Variantŝ2))*(Number of samples affected by all variants)/(Square root Length of the gene)

The purpose of this weighting was to value unique variants more highly than variants that were identified in multiple samples, to value genes more highly if they were found to be mutated in more samples, and to give a lower weighting to longer genes. The top 40 genes identified after this weighting procedure were then evaluated based on their function to produce a final list of 10 genes bearing mutations associated with bladder cancers (GENE LIST A, shown ranked according to predictive power in Table 7). GENE LIST A: TP53, NUP188, MUC16, CCDC168, KDM6A, SPTAN1, MLL2, ERBB3, ARID1A, and RB1.

TABLE 7
Diagnostic genes bearing bladder cancer-specific signature mutations
(GENE LIST A)

| Gene | | Entrez | Gene |
|---|---|---|---|
| # | Gene | Gene ID | Length* |
| 1 | TPJ3 | 7157 | 4858 |
| 2 | NUP188 | 23511 | 13570 |
| 3 | MUC16 | 94025 | 60267 |
| 4 | CCDC168 | 643677 | 22081 |
| 5 | KDM6A | 7403 | 11359 |
| 6 | SPTAN1 | 6709 | 18304 |
| 7 | MLL2 | 8085 | 27398 |
| 8 | ERBB3 | 2065 | 11108 |
| 9 | ARID1A | 8289 | 12201 |
| 10 | RB1 | 5925 | 9918 |

*As noted above, the “gene length” for a particular diagnostic gene includes the length, in basepairs, of the exons of that diagnostic gene, plus 100 base pairs from each (e.g., 5′ and 3′) end of any intervening sequences (e.g., introns) within that diagnostic gene, plus 100 base pairs of the untranslated regions abutting the 5′-most and 3′-most ends of the coding region. This length was used in the gene weighting calculations of the studies to detect the diagnostic genes of the disclosure, as described above.

The mutations identified in the genes in GENE LIST A are referred to herein as MUTATION PANEL A, and are specifically identified in Table 7.

### Example B

### Identification of a Second Panel of Gene Mutations Associated with Bladder Cancer (MUTATION PANEL B)

The tumor sample sequence dataset used for this analysis was the dataset generated in Example A, except that sequencing reads from only 45 samples were included as it was determined that 3 samples were inadvertently run twice, and those duplicate reads were removed. Hence, although 48 fresh-frozen human bladder tumor samples were processed through the Illumina Tru-seq Capture Protocol (Illumina, Inc.; San Diego, Calif.), and isolated tumor genomic DNA was sequenced using a massively parallel sequencing procedure employing an Illumina HiSeq2000 Sequencing Apparatus (Illumina, Inc.; San Diego, Calif.), the reads from 45 unique samples were analyzed as follows. The resulting reads were first aligned against HG19, and all discrepancies were cataloged. Discrepancies that occurred in 20% or greater of the reads were classified as variants. The variant calls were then compared with the dbSNP database; any variants that were present within dbSNP were excluded from further analysis. The remaining variant calls were then compared to the genomic sequences obtained from 106 unrelated human blood samples; if a variant call existed in more than 2 blood samples it was excluded. The purpose of comparing variant calls to the genomic sequences obtained from the 106 unrelated blood samples was two-fold: to supplement dbSNP in germline variant removal, and to remove any process-specific artifacts. The latter proved most relevant, as most of the variants removed following the comparison existed in all the blood genomic sequences and all the tumor genomic sequences. The remaining variants were then classified in 3 categories: synonymous, missense, and suspected deleterious. Synonymous variants have nucleic but no amino acid changes, and were ignored. Missense variant result in single amino acid changes, and may be detrimental to gene function. Suspected deleterious variants are stops (nonsense) or insertions and deletions that result in a frameshift; these both lead to truncations of the gene. The individual variants considered during further analysis of the candidate diagnostic genes in this study are shown in Table A as belonging to “Variant Class 1,” which comprised stop mutations, frameshift mutations and missense mutations. Variants excluded from these analyses are shown in Table A as belonging to “Variant Class 0,” which comprised synonymous mutations and splice site mutations. Genes were then weighted based upon their missense/suspected deleterious variants with the following formula, based on the Poisson distribution:

Weight(for a given gene)=(((p*l)̂n)/n!)*ê−(p*l)

### Where:

p=Probability of base being mutated, this was calculated based off of the observed mutation rate in this dataset.
l=Length of the gene.
n=number of unique variants.
e=Euler's number, ˜2.718.

The purpose of this weighting was to find the genes that had more mutations than expected for their gene length. In order to remove some spurious genes, the ratio of unique variants to total variants had to be greater than 0.3. All genes with a weighted score of less than 10⁻⁷ (i.e., less than 1×10E-7) were included in the list, excluding TTN, which yielded 99 genes bearing mutations associated with bladder cancers (GENE LIST B).

GENE LIST B: TP53, NUP188, XIRP2, PLCG2, KDM6A, CCDC168, KIAA1671, KPRP, OR5L2, SPTAN1, ERBB3, SRRM2, ARID1A, FOXM1, MUC16, ISG20L2, ZC3H7A, MYBPC2, AHNAK2, HSPBAP1, SYNE1, ZNF208, PLD1, SMC2, OR8I2, BTN2A2, MLL2, JMJD1C, SLC35G6, VCAN, VPS13D, VCX3B, ZNF705G, RBBP8, IGSF6, DOCKS, C9orf174, NPC1L1, PCDHGA9, ACTB, DNHD1, LYST, SCAF11, ZNF846, LOC100133128, DNAH17, DYNC1H1, ANK3, KIAA0100, STAG2, FLG, ZNF623, DCHS1, CARD6, KIF13A, HEATR1, MMP8, SCN9A, NLRP13, ZFHX4, ODZ3, TNP2, LOC653720, SPAG17, FAM75D1, UGT1A3, ABCA5, MFHAS1, CLCA4, PLXNA2, C2orf16, CEP95, ZNF217, HMCN1, UGGT1, CDRT15L2, FAT1, ZNF493, AKAP13, CDH13, CCL20, CPSF2, PSD4, FAM193A, XPOT, WWP1, GLDC, TNN, PDE4A, DNAJC10, COL12A1, NFL ITGA8, NPHP3, SAMD4A, COL21A1, NCKAP1L, MUC5B, and PCLO.

This list of genes bearing mutations associated with bladder cancers is also presented in Table 8, below, and the mutations identified in the genes in GENE LIST B are referred to herein as MUTATION PANEL B, and are specifically identified in Table B.

TABLE 8
Diagnostic genes bearing bladder cancer-specific signature mutations
(GENE LIST B)

| | | | | | | Samples | |
|---|---|---|---|---|---|---|---|
| | | | | Unique | Total | Affected | |
| Gene | | Entrez | Gene | Variant | Variant | by all | Weighted |
| # | Gene | Gene ID | Length* | Count | Counts | Variants | Score |
| 1 | TP53 | 7157 | 4858 | 12 | 13 | 12 | 1.55E−29 |
| 2 | NUP188 | 23511 | 13570 | 12 | 18 | 11 | 3.38E−24 |
| 3 | XIRP2 | 129446 | 15610 | 9 | 17 | 17 | 7.66E−17 |
| 4 | PLCG2 | 5336 | 10808 | 8 | 19 | 13 | 5.49E−16 |
| 5 | KDM6A | 7403 | 11359 | 8 | 8 | 8 | 8.15E−16 |
| 6 | CCDC168 | 643677 | 22081 | 9 | 9 | 9 | 1.69E−15 |
| 7 | KIAA1671 | 85379 | 12683 | 8 | 18 | 16 | 1.96E−15 |
| 8 | KPRP | 448834 | 2921 | 6 | 6 | 6 | 5.65E−15 |
| 9 | OR5L2 | 26338 | 1135 | 5 | 9 | 8 | 2.39E−14 |
| 10 | SPTAN1 | 6709 | 18304 | 8 | 8 | 5 | 3.60E−14 |
| 11 | ERBB3 | 2065 | 11108 | 7 | 7 | 6 | 1.13E−13 |
| 12 | SRRM2 | 23524 | 11953 | 7 | 7 | 5 | 1.89E−13 |
| 13 | ARID1A | 8289 | 12201 | 7 | 11 | 9 | 2.18E−13 |
| 14 | FOXM1 | 2305 | 5403 | 6 | 19 | 16 | 2.24E−13 |
| 15 | MUC16 | 94025 | 60267 | 10 | 18 | 15 | 3.14E−13 |
| 16 | ISG20L2 | 81875 | 2648 | 5 | 11 | 6 | 1.64E−12 |
| 17 | ZC3H7A | 29066 | 8286 | 6 | 6 | 2 | 2.88E−12 |
| 18 | MYBPC2 | 4606 | 8698 | 6 | 10 | 7 | 3.84E−12 |
| 19 | AHNAK2 | 113146 | 19572 | 7 | 22 | 20 | 5.77E−12 |
| 20 | HSPBAP1 | 79663 | 3455 | 5 | 15 | 15 | 6.18E−12 |
| 21 | SYNE1 | 23345 | 57157 | 9 | 15 | 14 | 7.56E−12 |
| 22 | ZNF208 | 7757 | 9884 | 6 | 11 | 9 | 8.23E−12 |
| 23 | PLD1 | 5337 | 10966 | 6 | 19 | 17 | 1.53E−11 |
| 24 | SMC2 | 10592 | 11297 | 6 | 6 | 3 | 1.82E−11 |
| 25 | OR8I2 | 120586 | 1132 | 4 | 7 | 5 | 2.40E−11 |
| 26 | BTN2A2 | 10385 | 5367 | 5 | 13 | 11 | 5.55E−11 |
| 27 | MLL2 | 8085 | 27398 | 7 | 7 | 6 | 5.87E−11 |
| 28 | JMJD1C | 221037 | 13810 | 6 | 6 | 5 | 6.02E−11 |
| 29 | SLC35G6 | 643664 | 1593 | 4 | 11 | 10 | 9.41E−11 |
| 30 | VCAN | 1462 | 15228 | 6 | 6 | 6 | 1.08E−10 |
| 31 | VPS13D | 55187 | 30041 | 7 | 7 | 5 | 1.11E−10 |
| 32 | VCX3B | 425054 | 1764 | 4 | 9 | 8 | 1.41E−10 |
| 33 | ZNF705G | 100131980 | 1920 | 4 | 4 | 4 | 1.98E−10 |
| 34 | RBBP8 | 5932 | 7416 | 5 | 5 | 5 | 2.77E−10 |
| 35 | IGSF6 | 10261 | 2222 | 4 | 5 | 5 | 3.55E−10 |
| 36 | DOCK9 | 23348 | 19186 | 6 | 6 | 4 | 4.23E−10 |
| 37 | C9orf174 | 100499483 | 354 | 3 | 4 | 4 | 6.01E−10 |
| 38 | NPC1L1 | 29881 | 8897 | 5 | 5 | 4 | 6.84E−10 |
| 39 | PCDHGA9 | 56107 | 2686 | 4 | 4 | 4 | 7.57E−10 |
| 40 | ACTB | 60 | 2747 | 4 | 4 | 2 | 8.28E−10 |
| 41 | DNHD1 | 144132 | 22844 | 6 | 13 | 12 | 1.19E−09 |
| 42 | LYST | 1130 | 23851 | 6 | 6 | 5 | 1.53E−09 |
| 43 | SCAF11 | 9169 | 10542 | 5 | 5 | 4 | 1.59E−09 |
| 44 | ZNF846 | 162993 | 3327 | 4 | 4 | 4 | 1.78E−09 |
| 45 | LOC100133128 | 100133128 | 620 | 3 | 5 | 5 | 3.23E−09 |
| 46 | DNAH17 | 8632 | 27298 | 6 | 6 | 6 | 3.39E−09 |
| 47 | DYNC1H1 | 1778 | 27420 | 6 | 7 | 6 | 3.48E−09 |
| 48 | ANK3 | 288 | 28629 | 6 | 6 | 5 | 4.48E−09 |
| 49 | KIAA0100 | 9703 | 13143 | 5 | 13 | 12 | 4.72E−09 |
| 50 | STAG2 | 10735 | 13183 | 5 | 5 | 4 | 4.80E−09 |
| 51 | FLG | 2312 | 13344 | 5 | 6 | 6 | 5.09E−09 |
| 52 | ZNF623 | 9831 | 4558 | 4 | 4 | 4 | 6.22E−09 |
| 53 | DCHS1 | 8642 | 14137 | 5 | 5 | 3 | 6.77E−09 |
| 54 | CARD6 | 84674 | 4694 | 4 | 6 | 6 | 7.00E−09 |
| 55 | KIF13A | 63971 | 14303 | 5 | 5 | 5 | 7.17E−09 |
| 56 | HEATR1 | 55127 | 14505 | 5 | 5 | 5 | 7.69E−09 |
| 57 | MMP8 | 4317 | 4933 | 4 | 4 | 3 | 8.53E−09 |
| 58 | SCN9A | 6335 | 15069 | 5 | 5 | 5 | 9.28E−09 |
| 59 | NLRP13 | 126204 | 5346 | 4 | 4 | 4 | 1.17E−08 |
| 60 | ZFHX4 | 79776 | 16147 | 5 | 6 | 6 | 1.31E−08 |
| 61 | ODZ3 | 55714 | 16176 | 5 | 5 | 4 | 1.32E−08 |
| 62 | TNP2 | 7142 | 995 | 3 | 7 | 5 | 1.33E−08 |
| 63 | LOC653720 | 653720 | 5565 | 4 | 4 | 2 | 1.38E−08 |
| 64 | SPAG17 | 200162 | 16454 | 5 | 5 | 5 | 1.43E−08 |
| 65 | FAM75D1 | 389763 | 5629 | 4 | 4 | 4 | 1.44E−08 |
| 66 | UGT1A3 | 54659 | 1066 | 3 | 3 | 3 | 1.64E−08 |
| 67 | ABCA5 | 23461 | 16948 | 5 | 5 | 5 | 1.66E−08 |
| 68 | MFHAS1 | 9258 | 5852 | 4 | 11 | 9 | 1.68E−08 |
| 69 | CLCA4 | 22802 | 5937 | 4 | 4 | 4 | 1.78E−08 |
| 70 | PLXNA2 | 5362 | 17765 | 5 | 5 | 4 | 2.09E−08 |
| 71 | C2orf16 | 84226 | 6400 | 4 | 4 | 4 | 2.40E−08 |
| 72 | CEP95 | 90799 | 6567 | 4 | 5 | 5 | 2.66E−08 |
| 73 | ZNF217 | 7764 | 6630 | 4 | 4 | 3 | 2.76E−08 |
| 74 | HMCN1 | 83872 | 39142 | 6 | 6 | 6 | 2.80E−08 |
| 75 | UGGT1 | 56886 | 19121 | 5 | 5 | 4 | 3.00E−08 |
| 76 | CDRT15L2 | 256223 | 1386 | 3 | 6 | 5 | 3.59E−08 |
| 77 | FAT1 | 2195 | 19980 | 5 | 5 | 5 | 3.72E−08 |
| 78 | ZNF493 | 284443 | 7280 | 4 | 6 | 3 | 4.00E−08 |
| 79 | AKAP13 | 11214 | 21025 | 5 | 5 | 4 | 4.78E−08 |
| 80 | CDH13 | 1012 | 7714 | 4 | 4 | 3 | 5.04E−08 |
| 81 | CCL20 | 6364 | 1638 | 3 | 3 | 2 | 5.92E−08 |
| 82 | CPSF2 | 53981 | 8135 | 4 | 4 | 4 | 6.22E−08 |
| 83 | PSD4 | 23550 | 8261 | 4 | 4 | 1 | 6.61E−08 |
| 84 | FAM193A | 8603 | 8372 | 4 | 4 | 3 | 6.97E−08 |
| 85 | XPOT | 11260 | 8684 | 4 | 4 | 2 | 8.06E−08 |
| 86 | WWP1 | 11059 | 8695 | 4 | 6 | 2 | 8.10E−08 |
| 87 | GLDC | 2731 | 8759 | 4 | 11 | 6 | 8.34E−08 |
| 88 | TNN | 63923 | 8789 | 4 | 12 | 11 | 8.45E−08 |
| 89 | PDE4A | 5141 | 8907 | 4 | 10 | 9 | 8.91E−08 |
| 90 | DNAJC10 | 54431 | 8969 | 4 | 4 | 3 | 9.16E−08 |
| 91 | COL12A1 | 1303 | 24090 | 5 | 5 | 5 | 9.32E−08 |
| 92 | NF1 | 4763 | 24550 | 5 | 13 | 7 | 1.02E−07 |
| 93 | ITGA8 | 8516 | 9231 | 4 | 4 | 4 | 1.03E−07 |
| 94 | NPHP3 | 27031 | 1978 | 3 | 6 | 6 | 1.04E−07 |
| 95 | SAMD4A | 23034 | 9419 | 4 | 8 | 8 | 1.11E−07 |
| 96 | COL21A1 | 81578 | 9746 | 4 | 4 | 4 | 1.27E−07 |
| 97 | NCKAP1L | 3071 | 9933 | 4 | 4 | 4 | 1.37E−07 |
| 98 | MUC5B | 727897 | 26870 | 5 | 16 | 13 | 1.59E−07 |
| 99 | PCLO | 27445 | 27278 | 5 | 5 | 5 | 1.71E−07 |

*As noted above, the “gene length” for a particular diagnostic gene includes the length, in basepairs, of the exons of that diagnostic gene, plus 100 base pairs from each (e.g., 5′ and 3′) end of any intervening sequences (e.g., introns) within that diagnostic gene, plus 100 base pairs of the untranslated regions abutting the 5′-most and 3′-most ends of the coding region. This length was used in the gene weighting calculations of the studies to detect the diagnostic genes of the disclosure, as described above.

### Example C

### Identification of a Third Panel of Gene Mutations Associated with Bladder Cancer (MUTATION PANEL C)

The tumor sample sequence dataset used for this analysis was the dataset generated in Example A, except that sequencing reads from only 45 samples were included as it was determined that 3 samples were inadvertently run twice, and those duplicate reads were removed. Hence, although 48 fresh-frozen human bladder tumor samples were processed through the Illumina Tru-seq Capture Protocol (Illumina, Inc.; San Diego, Calif.), and isolated tumor genomic DNA was sequenced using a massively parallel sequencing procedure employing an Illumina HiSeq2000 Sequencing Apparatus (Illumina, Inc.; San Diego, Calif.), the reads from 45 unique samples were analyzed as follows. The resulting reads were first aligned against HG19, and all discrepancies were cataloged. Discrepancies that occurred in 20% or greater of the reads were classified as variants. The variant calls were then compared with the dbSNP database; any variants that were present within dbSNP were excluded from further analysis. The remaining variant calls were then compared to the genomic sequences obtained from 106 unrelated human blood samples; if a variant call existed in more than 2 blood samples it was excluded. The purpose of comparing variant calls to the genomic sequences obtained from the 106 unrelated blood samples was two-fold: to supplement dbSNP in germline variant removal, and to remove any process-specific artifacts. The latter proved most relevant, as most of the variants removed following the comparison existed in all the blood genomic sequences and all the tumor genomic sequences. The remaining variants were then classified in 3 categories: synonymous, missense, and suspected deleterious. Synonymous variants have nucleic but no amino acid changes, and were ignored. Missense variant result in single amino acid changes, and may be detrimental to gene function. Suspected deleterious variants are stops (nonsense) or insertions and deletions that result in a frameshift, which both lead to truncations of the gene, as well as mutations that likely adversely affect the correct splicing of exons of the encoded gene product. The individual variants considered during further analysis of the candidate diagnostic genes in this study are shown in Table 9 as belonging to “Variant Class 1,” which comprised stop mutations, frameshift mutations, missense mutations and splice site mutations. Variants excluded from these analyses are shown in Table 9 as belonging to “Variant Class 0,” which comprised only synonymous mutations. Genes were then weighted based upon their missense/suspected deleterious variants with the following formula, based on the Poisson distribution:

Weight(for a given gene)=(((p*l)̂n)/n!)*ê−(p*l)

### Where:

p=Probability of base being mutated, this was calculated based off of the observed mutation rate in this dataset.
l=Length of the gene.
n=number of unique variants.
e=Euler's number, ˜2.718.

The purpose of this weighting was to find the genes that had more mutations than expected for their gene length. In order to remove some spurious genes, the ratio of unique variants to total variants had to be greater than 0.3. All genes with a weighted score of less than 10⁻⁷ (i.e., less than 1×10E-7) were included in the list, excluding TTN, which yielded 99 genes bearing mutations associated with bladder cancers (GENE LIST C).

GENE LIST C: TP53, NUP188, XIRP2, PLCG2, FOXM1, KDM6A, ARID1A, CCDC168, KIAA1671, KPRP, MUC16, OR5L2, SPTAN1, ERBB3, SRRM2, SNRNP200, ISG20L2, ZC3H7A, MYBPC2, AHNAK2, HSPBAP1, SYNE1, ZNF208, PLD1, SMC2, OR8I2, STAG2, BTN2A2, MLL2, JMJD1C, SLC35G6, VCAN, VPS13D, VCX3B, ZNF705G, RBBP8, IGSF6, DOCKS, C9orf174, NPC1L1, PCDHGA9, ACTB, DNHD1, LYST, SCAF11, ZNF846, NF1, CACNA2D3, LAPTM4B, LOC100133128, PCLO, DNAH17, DYNC1H1, ANK3, KIAA0100, FLG, ABCB5, POLR3C, ZNF623, DCHS1, CARD6, KIF13A, HEATR1, WDR6, MMP8, SCN9A, NLRP13, ZFHX4, ODZ3, TNP2, LOC653720, SPAG17, FAM75D1, UGT1A3, ABCA5, MFHAS1, CLCA4, CRTAC1, CHD6, PLXNA2, RYR2, C2orf16, CEP95, ZNF217, HMCN1, UGGT1, CDRT15L2, FAT1, ZNF493, AKAP13, CDH13, CCL20, CPSF2, PSD4, FAM193A, XPOT, WWP1, GLDC, and TNN.

This list of genes bearing mutations associated with bladder cancers is also presented in Table 9, below, and the mutations identified in the genes in GENE LIST C are referred to herein as MUTATION PANEL C, and are specifically identified in Table C.

TABLE 9
Diagnostic genes bearing bladder cancer-specific signature mutations
(GENE LIST C)

| | | | | | | Samples | |
|---|---|---|---|---|---|---|---|
| | | | | Unique | Total | Affected | |
| Gene | | Entrez | Gene | Variant | Variant | by all | Weighted |
| # | Gene | Gene ID | Length* | Count | Counts | Variants | Score |
| 1 | TP53 | 7157 | 4858 | 13 | 14 | 13 | 2.52E−32 |
| 2 | NUP188 | 23511 | 13570 | 12 | 18 | 11 | 3.38E−24 |
| 3 | XIRP2 | 129446 | 15610 | 10 | 18 | 18 | 5.18E−19 |
| 4 | PLCG2 | 5336 | 10808 | 8 | 19 | 13 | 5.49E−16 |
| 5 | FOXM1 | 2305 | 5403 | 7 | 20 | 17 | 7.49E−16 |
| 6 | KDM6A | 7403 | 11359 | 8 | 8 | 8 | 8.15E−16 |
| 7 | ARID1A | 8289 | 12201 | 8 | 12 | 10 | 1.44E−15 |
| 8 | CCDC168 | 643677 | 22081 | 9 | 9 | 9 | 1.69E−15 |
| 9 | KIAA1671 | 85379 | 12683 | 8 | 18 | 16 | 1.96E−15 |
| 10 | KPRP | 448834 | 2921 | 6 | 6 | 6 | 5.65E−15 |
| 11 | MUC16 | 94025 | 60267 | 11 | 19 | 16 | 7.46E−15 |
| 12 | OR5L2 | 26338 | 1135 | 5 | 9 | 8 | 2.39E−14 |
| 13 | SPTAN1 | 6709 | 18304 | 8 | 8 | 5 | 3.60E−14 |
| 14 | ERBB3 | 2065 | 11108 | 7 | 7 | 6 | 1.13E−13 |
| 15 | SRRM2 | 23524 | 11953 | 7 | 7 | 5 | 1.89E−13 |
| 16 | SNRNP200 | 23020 | 15227 | 7 | 22 | 19 | 1.01E−12 |
| 17 | ISG20L2 | 81875 | 2648 | 5 | 11 | 6 | 1.64E−12 |
| 18 | ZC3H7A | 29066 | 8286 | 6 | 6 | 2 | 2.88E−12 |
| 19 | MYBPC2 | 4606 | 8698 | 6 | 10 | 7 | 3.84E−12 |
| 20 | AHNAK2 | 113146 | 19572 | 7 | 22 | 20 | 5.77E−12 |
| 21 | HSPBAP1 | 79663 | 3455 | 5 | 15 | 15 | 6.18E−12 |
| 22 | SYNE1 | 23345 | 57157 | 9 | 15 | 14 | 7.56E−12 |
| 23 | ZNF208 | 7757 | 9884 | 6 | 11 | 9 | 8.23E−12 |
| 24 | PLD1 | 5337 | 10966 | 6 | 19 | 17 | 1.53E−11 |
| 25 | SMC2 | 10592 | 11297 | 6 | 6 | 3 | 1.82E−11 |
| 26 | OR8I2 | 120586 | 1132 | 4 | 7 | 5 | 2.40E−11 |
| 27 | STAG2 | 10735 | 13183 | 6 | 6 | 5 | 4.57E−11 |
| 28 | BTN2A2 | 10385 | 5367 | 5 | 13 | 11 | 5.55E−11 |
| 29 | MLL2 | 8085 | 27398 | 7 | 7 | 6 | 5.87E−11 |
| 30 | JMJD1C | 221037 | 13810 | 6 | 6 | 5 | 6.02E−11 |
| 31 | SLC35G6 | 643664 | 1593 | 4 | 11 | 10 | 9.41E−11 |
| 32 | VCAN | 1462 | 15228 | 6 | 6 | 6 | 1.08E−10 |
| 33 | VPS13D | 55187 | 30041 | 7 | 7 | 5 | 1.11E−10 |
| 34 | VCX3B | 425054 | 1764 | 4 | 9 | 8 | 1.41E−10 |
| 35 | ZNF705G | 100131980 | 1920 | 4 | 4 | 4 | 1.98E−10 |
| 36 | RBBP8 | 5932 | 7416 | 5 | 5 | 5 | 2.77E−10 |
| 37 | IGSF6 | 10261 | 2222 | 4 | 5 | 5 | 3.55E−10 |
| 38 | DOCK9 | 23348 | 19186 | 6 | 6 | 4 | 4.23E−10 |
| 39 | C9orf174 | 100499483 | 354 | 3 | 4 | 4 | 6.01E−10 |
| 40 | NPC1L1 | 29881 | 8897 | 5 | 5 | 4 | 6.84E−10 |
| 41 | PCDHGA9 | 56107 | 2686 | 4 | 4 | 4 | 7.57E−10 |
| 42 | ACTB | 60 | 2747 | 4 | 4 | 2 | 8.28E−10 |
| 43 | DNHD1 | 144132 | 22844 | 6 | 13 | 12 | 1.19E−09 |
| 44 | LYST | 1130 | 23851 | 6 | 6 | 5 | 1.53E−09 |
| 45 | SCAF11 | 9169 | 10542 | 5 | 5 | 4 | 1.59E−09 |
| 46 | ZNF846 | 162993 | 3327 | 4 | 4 | 4 | 1.78E−09 |
| 47 | NF1 | 4763 | 24550 | 6 | 18 | 10 | 1.81E−09 |
| 48 | CACNA2D3 | 55799 | 11097 | 5 | 6 | 5 | 2.05E−09 |
| 49 | LAPTM4B | 55353 | 3631 | 4 | 4 | 1 | 2.52E−09 |
| 50 | LOC100133128 | 100133128 | 620 | 3 | 5 | 5 | 3.23E−09 |
| 51 | PCLO | 27445 | 27278 | 6 | 6 | 6 | 3.37E−09 |
| 52 | DNAH17 | 8632 | 27298 | 6 | 6 | 6 | 3.39E−09 |
| 53 | DYNC1H1 | 1778 | 27420 | 6 | 7 | 6 | 3.48E−09 |
| 54 | ANK3 | 288 | 28629 | 6 | 6 | 5 | 4.48E−09 |
| 55 | KIAA0100 | 9703 | 13143 | 5 | 13 | 12 | 4.72E−09 |
| 56 | FLG | 2312 | 13344 | 5 | 6 | 6 | 5.09E−09 |
| 57 | ABCB5 | 340273 | 13391 | 5 | 5 | 5 | 5.18E−09 |
| 58 | POLR3C | 10623 | 4546 | 4 | 4 | 4 | 6.16E−09 |
| 59 | ZNF623 | 9831 | 4558 | 4 | 4 | 4 | 6.22E−09 |
| 60 | DCHS1 | 8642 | 14137 | 5 | 5 | 3 | 6.77E−09 |
| 61 | CARD6 | 84674 | 4694 | 4 | 6 | 6 | 7.00E−09 |
| 62 | KIF13A | 63971 | 14303 | 5 | 5 | 5 | 7.17E−09 |
| 63 | HEATR1 | 55127 | 14505 | 5 | 5 | 5 | 7.69E−09 |
| 64 | WDR6 | 11180 | 4895 | 4 | 6 | 6 | 8.27E−09 |
| 65 | MMP8 | 4317 | 4933 | 4 | 4 | 3 | 8.53E−09 |
| 66 | SCN9A | 6335 | 15069 | 5 | 5 | 5 | 9.28E−09 |
| 67 | NLRP13 | 126204 | 5346 | 4 | 4 | 4 | 1.17E−08 |
| 68 | ZFHX4 | 79776 | 16147 | 5 | 6 | 6 | 1.31E−08 |
| 69 | ODZ3 | 55714 | 16176 | 5 | 5 | 4 | 1.32E−08 |
| 70 | TNP2 | 7142 | 995 | 3 | 7 | 5 | 1.33E−08 |
| 71 | LOC653720 | 653720 | 5565 | 4 | 4 | 2 | 1.38E−08 |
| 72 | SPAG17 | 200162 | 16454 | 5 | 5 | 5 | 1.43E−08 |
| 73 | FAM75D1 | 389763 | 5629 | 4 | 4 | 4 | 1.44E−08 |
| 74 | UGT1A3 | 54659 | 1066 | 3 | 3 | 3 | 1.64E−08 |
| 75 | ABCA5 | 23461 | 16948 | 5 | 5 | 5 | 1.66E−08 |
| 76 | MFHAS1 | 9258 | 5852 | 4 | 11 | 9 | 1.68E−08 |
| 77 | CLCA4 | 22802 | 5937 | 4 | 4 | 4 | 1.78E−08 |
| 78 | CRTAC1 | 55118 | 5948 | 4 | 6 | 6 | 1.79E−08 |
| 79 | CHD6 | 84181 | 17737 | 5 | 7 | 5 | 2.07E−08 |
| 80 | PLXNA2 | 5362 | 17765 | 5 | 5 | 4 | 2.09E−08 |
| 81 | RYR2 | 6262 | 37260 | 6 | 6 | 5 | 2.10E−08 |
| 82 | C2orf16 | 84226 | 6400 | 4 | 4 | 4 | 2.40E−08 |
| 83 | CEP95 | 90799 | 6567 | 4 | 5 | 5 | 2.66E−08 |
| 84 | ZNF217 | 7764 | 6630 | 4 | 4 | 3 | 2.76E−08 |
| 85 | HMCN1 | 83872 | 39142 | 6 | 6 | 6 | 2.80E−08 |
| 86 | UGGT1 | 56886 | 19121 | 5 | 5 | 4 | 3.00E−08 |
| 87 | CDRT15L2 | 256223 | 1386 | 3 | 6 | 5 | 3.59E−08 |
| 88 | FAT1 | 2195 | 19980 | 5 | 5 | 5 | 3.72E−08 |
| 89 | ZNF493 | 284443 | 7280 | 4 | 6 | 3 | 4.00E−08 |
| 90 | AKAP13 | 11214 | 21025 | 5 | 5 | 4 | 4.78E−08 |
| 91 | CDH13 | 1012 | 7714 | 4 | 4 | 3 | 5.04E−08 |
| 92 | CCL20 | 6364 | 1638 | 3 | 3 | 2 | 5.92E−08 |
| 93 | CPSF2 | 53981 | 8135 | 4 | 4 | 4 | 6.22E−08 |
| 94 | PSD4 | 23550 | 8261 | 4 | 4 | 1 | 6.61E−08 |
| 95 | FAM193A | 8603 | 8372 | 4 | 4 | 3 | 6.97E−08 |
| 96 | XPOT | 11260 | 8684 | 4 | 4 | 2 | 8.06E−08 |
| 97 | WWP1 | 11059 | 8695 | 4 | 6 | 2 | 8.10E−08 |
| 98 | GLDC | 2731 | 8759 | 4 | 11 | 6 | 8.34E−08 |
| 99 | TNN | 63923 | 8789 | 4 | 12 | 11 | 8.45E−08 |

*As noted above, the “gene length” for a particular diagnostic gene includes the length, in basepairs, of the exons of that diagnostic gene, plus 100 base pairs from each (e.g., 5′ and 3′) end of any intervening sequences (e.g., introns) within that diagnostic gene, plus 100 base pairs of the untranslated regions abutting the 5′-most and 3′-most ends of the coding region. This length was used in the gene weighting calculations of the studies to detect the diagnostic genes of the disclosure, as described above.

### Example D

### Identification of a Fourth Panel of Gene Mutations Associated with Bladder Cancer (MUTATION PANEL D)

The tumor sample sequence dataset used for this analysis was the dataset generated in Example A, except that sequencing reads from only 45 samples were included as it was determined that 3 samples were inadvertently run twice, and those duplicate reads were removed. Also, 8 new samples were included that were processed through the Illumina Nextera Protocol (Illumina, Inc.; San Diego, Calif.), and isolated tumor genomic DNA was sequenced using a massively parallel sequencing procedure employing a HiSeq2500 Sequencing Apparatus (Illumina, Inc.; San Diego, Calif.). Hence, a total of 53 distinct fresh-frozen human bladder tumor samples were processed either through the Illumina Tru-seq Capture Protocol, or the Illumina Nextera Protocol (Illumina, Inc.; San Diego, Calif.), and isolated tumor genomic DNA was sequenced using a massively parallel sequencing procedure employing either an Illumina HiSeq2000 or HiSeq2500 Sequencing Apparatus (Illumina, Inc.; San Diego, Calif.), and the sequencing reads from all 53 unique samples were analyzed as follows.

The resulting reads were first aligned against HG19, and all discrepancies were cataloged. Discrepancies that occurred in 20% or greater of the reads were classified as variants. The variant calls were then compared with the dbSNP database; any variants that were present within dbSNP were excluded from further analysis. The remaining variant calls were then compared to the genomic sequences obtained from 106 unrelated human blood samples; if a variant call existed in more than 2 blood samples it was excluded. The purpose of comparing variant calls to the genomic sequences obtained from the 106 unrelated blood samples was two-fold: to supplement dbSNP in germline variant removal, and to remove any process-specific artifacts. The latter proved most relevant, as most of the variants removed following the comparison existed in all the blood genomic sequences and all the tumor genomic sequences.

The remaining variants were further analyzed for their effect on the encoded protein and mRNA transcript encoding the protein. Variants causing a truncation of the encoded protein through the introduction of stop codons (e.g., nonsense mutations), insertion or deletion mutations that result in a shift in the reading frame (e.g., frameshift mutations), and intronic mutations that potentially alter transcript splicing were categorized as “class 1” variants, having a higher chance in causing a change in function. Variants that resulted in missense mutations, insertions or deletions that maintained the reading frame, and intronic mutations that likely would not alter transcript splicing were categorized as “class 0” variants, having a lower chance of causing a functional change. Unlike with the previous Examples, variants of both classes (class 1 and class 0) were included in the gene weighting calculations. Additionally, as described below, the weighting protocol for EXAMPLE D differed from the weighting protocol for the previous Examples.

In addition to the 105 diagnostic genes containing signature mutations revealed through comparison of tumor exomes with a reference human genome, four additional genes containing mutations previously identified as indicators of bladder cancer were included in the diagnostic methods of this example (See Kompier, L C et al. Bladder cancer: novel molecular characteristics, diagnostics, and therapeutic indications. Urol. Oncol. 2010 January-February; 28(1):91-6 and Huang, F W, et al. Highly recurrent TERT promoter mutations in human melanoma. Science 2013 Feb. 22; 339(6122):957-9). The four additional genes are FIBROBLAST GROWTH FACTOR RECEPTOR 3 (FGFR3; (GRCh37): 4:1,795,038-1,810,598; OMIM 134934); PHOSPHATIDYL-INOSITOL 3-KINASE, CATALYTIC, ALPHA (PI3KCA; (GRCh37): 3:178,866,310-178,952,499; OMIM: 171834); V-KI-RAS2 KIRSTEN RAT SARCOMA VIRAL ONCOGENE HOMOLOG (KRAS; (GRCh37): 12:25,358,179-25,403,869; OMIM: 190070); and TELOMERASE REVERSE TRANSCRIPTASE (TERT; (GRCh37): 5:1,253,281-1,295,177; OMIM: 187270). The first three of these additional genes (i.e., FGFR3, PI3KCA, and KRAS) were treated in the same manner as the 105 diagnostic containing signature mutations revealed through comparison of tumor exomes with a reference human genome.

In addition, all 53 human bladder cancer tumors were screened for the presence or absence of two C to T transitions in the promoter of the TERT gene. The first of these C to T transitions occurs at genomic coordinate Chr5:1,295,228 (GRCh37) and is also referred to as −124G>A or C228T, and the second occurs at genomic coordinate Chr5:1,295,250 (GRCh37) and is also referred to as −146G>A or C250T. (OMIM 187270) Both C228T and C250T generate de novo consensus binding motifs for E-twenty-six (ETS) transcription factors that increase transcriptional activity from the TERT promoter and result in increased expression of telomerase reverse transcriptase, the protein encoded by the TERT gene. (See: Huang, F W, et al. Highly recurrent TERT promoter mutations in human melanoma. Science 2013 Feb. 22; 339(6122):957-9.)

Screening of the TERT gene promoter was done by manual Sanger sequencing. Out of the 53 bladder cancer tumors, 21 were found to carry the mutation C228T, and 3 were found to carry the mutation C250T (see: Table D). The other 29 exome sequences had the wild-type sequence C228/C250. When either of the two TERT promoter mutations (C228T and C250T) was present, they were assigned to variant class 2, since these mutations are known to result in increased transcription of the TERT gene, e.g., they are “activating mutations.”

Unlike in the prior EXAMPLES, the individual variants considered during further analysis of the candidate diagnostic genes in this study are shown in Table D as belonging to either variant class 1 or variant class 0. Genes were then weighted based upon the following formula, which is based on the Poisson distribution:

Weight(for a given gene)=(((p*l)̂n)/n!)*ê−(p*l)

### Where:

p=Probability of base being mutated, this was calculated based off of the observed mutation rate in this dataset.
l=Length of the gene.
n=number of unique variants, wherein each unique variant is deweighted by the number of samples in which it appears.
e=Euler's number, ˜2.718.

The purpose of this weighting was to find the genes that had more mutations than expected for their length.

In this analysis, each variant (n) is deweighted by the number of samples in which it appears, so that if a variant is unique to 1 sample, it is weighted as 1. If a variant appears in 2 samples, it would be ½, etc. So, in Examples A-C, above, if a gene had 2 variants, it would have an “n” of 2, even if one variant was unique and the other appeared in 10 samples. Whereas, using the deweighting technique applied in this Example, the gene's “n” would be 1+( 1/10) or 1.1.

In order to remove some spurious genes, the ratio of unique variants to total variants had to be greater than 0.3. All genes with a weighted score of less than 10⁻⁷ (i.e., less than 1×10E-7) were included in the list, as were PIK3CA, KRAS, FGFR3 and TERT, which were added because mutations in these four genes had previously been associated with bladder cancer, to yield 109 genes bearing signature mutations associated with bladder cancers (GENE LIST D).

GENE LIST D: TP53, MLL2, ARID1A, KDM6A, PCLO, C10orf71, ZFHX4, PCNXL2, XIRP2, FOXM1, ODZ3, DNAH17, FLG, PLEC, RP1L1, LOC100130830, OBSCN, NLRP13, AGRN, SPTAN1, PCDHGA2, KPRP, RBBP8, PCDHGA9, OR2T4, AHNAK2, MUC16, RNF111, COL6A1, PCDH8, NACAD, UNC93B1, WDR6, ZRANB3, SRRM2, TMEM175, AKAP13, INPP5D, KIF7, CHD8, NEB, ZSCAN5D, CCDC40, RB1, CAMTA2, KIAA1683, HSPBAP1, GYG2, VPS13D, GLIS2, SUV420H1, JMJD1C, MFHAS1, STAG2, SYNE2, GIMAP6, NUP188, KIF21A, MAGI1, PLXNA2, SCN5A, PLCL2, LIFR, SPEN, KALRN, MAGEC1, LRP1B, C16orf96, SMC2, C7orf58, KNTC1, AZU1, RBM10, PCDHA2, CLCA4, MAST4, ATP2C2, ACTB, INPP5F, USH2A, IGSF6, GPR98, NPHP3, ZNF469, CPSF1, TONSL, FAN1, IQSEC2, APOB, RSF1, NBEA, MIR205HG, ZFP36L1, POLE, DST, NVL, ZNFX1, FREM2, PCDHGA5, RECQL5, MLL, HRAS, ERBB2, ERBB3, MLL3, PI3KCA, KRAS, FGFR3 and TERT.

This list of genes bearing mutations associated with bladder cancers is also presented in Table 10, below, and the mutations identified in the genes in GENE LIST D are referred to herein as MUTATION PANEL D, and are specifically identified in Table D.

TABLE 10
Diagnostic genes bearing bladder cancer-specific signature mutations
(GENE LIST D)

| | | | | | | Samples | |
|---|---|---|---|---|---|---|---|
| | | | | Weighted | Total | Affected | |
| Gene | | Entrez | Gene | Variant | Variant | by all | Weighted |
| # | Gene | Gene ID | Length* | Count** | Count | Variants | Score |
| 1 | TP53 | 7157 | 4858 | 26.333333 | 29 | 25 | 3.85E−62 |
| 2 | MLL2 | 8085 | 27398 | 24 | 24 | 16 | 1.99E−31 |
| 3 | ARID1A | 8289 | 12201 | 13.522222 | 60 | 47 | 2.09E−22 |
| 4 | KDM6A | 7403 | 11359 | 10.5 | 12 | 11 | 8.09E−20 |
| 5 | PCLO | 27445 | 27278 | 12 | 12 | 11 | 6.26E−19 |
| 6 | C10orf71 | 118461 | 5883 | 8 | 8 | 8 | 4.32E−18 |
| 7 | ZFHX4 | 79776 | 16147 | 9.5 | 11 | 11 | 8.77E−18 |
| 8 | PCNXL2 | 80003 | 13423 | 9 | 9 | 9 | 1.99E−17 |
| 9 | XIRP2 | 129446 | 15610 | 9.2 | 14 | 14 | 2.84E−17 |
| 10 | FOXM1 | 2305 | 5403 | 8.0625 | 24 | 20 | 1.41E−15 |
| 11 | ODZ3 | 55714 | 16176 | 10 | 10 | 8 | 3.12E−15 |
| 12 | DNAH17 | 8632 | 27298 | 10 | 10 | 9 | 5.10E−15 |
| 13 | FLG | 2312 | 13344 | 8.333333 | 11 | 10 | 1.35E−14 |
| 14 | PLEC | 5339 | 22434 | 12.077922 | 66 | 43 | 1.98E−14 |
| 15 | RP1L1 | 94137 | 8774 | 8.0625 | 24 | 20 | 3.63E−14 |
| 16 | LOC100130830 | 100130830 | 1934 | 6.946078 | 52 | 38 | 6.10E−14 |
| 17 | OBSCN | 84033 | 45895 | 11.019231 | 63 | 52 | 1.82E−13 |
| 18 | NLRP13 | 126204 | 5346 | 6 | 6 | 6 | 2.10E−13 |
| 19 | AGRN | 375790 | 12458 | 9.185535 | 74 | 53 | 3.40E−13 |
| 20 | SPTAN1 | 6709 | 18304 | 10 | 10 | 7 | 4.79E−13 |
| 21 | PCDHGA2 | 56113 | 2856 | 6 | 6 | 5 | 1.20E−12 |
| 22 | KPRP | 448834 | 2921 | 6 | 6 | 5 | 1.34E−12 |
| 23 | RBBP8 | 5932 | 7416 | 6 | 6 | 6 | 1.48E−12 |
| 24 | PCDHGA9 | 56107 | 2686 | 5 | 5 | 5 | 1.76E−12 |
| 25 | OR2T4 | 127074 | 1246 | 5.527027 | 50 | 39 | 1.94E−12 |
| 26 | AHNAK2 | 113146 | 19572 | 8.03125 | 40 | 35 | 2.42E−12 |
| 27 | MUC16 | 94025 | 60267 | 11.625 | 21 | 16 | 2.81E−12 |
| 28 | RNF111 | 54778 | 8324 | 6 | 6 | 6 | 2.96E−12 |
| 29 | COL6A1 | 1291 | 10407 | 7.026316 | 45 | 39 | 3.63E−12 |
| 30 | PCDH8 | 5100 | 4573 | 6.282258 | 41 | 34 | 3.63E−12 |
| 31 | NACAD | 23148 | 5958 | 6.066667 | 21 | 19 | 4.36E−12 |
| 32 | UNC93B1 | 81622 | 4453 | 6.469231 | 67 | 53 | 4.49E−12 |
| 33 | WDR6 | 11180 | 4895 | 5.333333 | 8 | 8 | 5.45E−12 |
| 34 | ZRANB3 | 84083 | 7877 | 6.5 | 8 | 7 | 6.05E−12 |
| 35 | SRRM2 | 23524 | 11953 | 8 | 8 | 6 | 6.54E−12 |
| 36 | TMEM175 | 84286 | 3949 | 5.361111 | 18 | 17 | 6.72E−12 |
| 37 | AKAP13 | 11214 | 21025 | 8.052632 | 27 | 23 | 7.33E−12 |
| 38 | INPP5D | 3635 | 10174 | 6 | 6 | 6 | 9.78E−12 |
| 39 | KIF7 | 374654 | 8087 | 6.333333 | 9 | 8 | 1.02E−11 |
| 40 | CHD8 | 57680 | 15509 | 6.5 | 8 | 8 | 1.20E−11 |
| 41 | NEB | 4703 | 60665 | 9 | 9 | 9 | 1.27E−11 |
| 42 | ZSCAN5D | 646698 | 2299 | 10.732471 | 143 | 54 | 1.33E−11 |
| 43 | CCDC40 | 55036 | 8034 | 6.667832 | 32 | 26 | 2.06E−11 |
| 44 | RB1 | 5925 | 9918 | 6.5 | 8 | 7 | 2.23E−11 |
| 45 | CAMTA2 | 23125 | 9295 | 6.333333 | 9 | 8 | 2.23E−11 |
| 46 | KIAA1683 | 80726 | 5239 | 6 | 6 | 5 | 2.46E−11 |
| 47 | HSPBAP1 | 79663 | 3455 | 5.071429 | 29 | 27 | 2.47E−11 |
| 48 | GYG2 | 8908 | 5705 | 5.2 | 10 | 10 | 2.54E−11 |
| 49 | VPS13D | 55187 | 30041 | 9 | 9 | 7 | 2.72E−11 |
| 50 | GLIS2 | 84662 | 4662 | 5 | 5 | 5 | 2.75E−11 |
| 51 | SUV420H1 | 51111 | 7894 | 6.018519 | 60 | 54 | 2.90E−11 |
| 52 | JMJD1C | 221037 | 13810 | 7 | 7 | 6 | 2.93E−11 |
| 53 | MFHAS1 | 9258 | 5852 | 6.25 | 31 | 25 | 3.01E−11 |
| 54 | STAG2 | 10735 | 13183 | 7.5 | 9 | 7 | 3.19E−11 |
| 55 | SYNE2 | 23224 | 44698 | 11.632576 | 51 | 32 | 3.55E−11 |
| 56 | GIMAP6 | 474344 | 4244 | 5.076923 | 18 | 17 | 4.51E−11 |
| 57 | NUP188 | 23511 | 13570 | 10.2 | 15 | 8 | 4.84E−11 |
| 58 | KIF21A | 55605 | 13964 | 6 | 6 | 6 | 6.43E−11 |
| 59 | MAGI1 | 9223 | 13986 | 6 | 6 | 6 | 6.49E−11 |
| 60 | PLXNA2 | 5362 | 17765 | 8 | 8 | 6 | 6.92E−11 |
| 61 | SCN5A | 6331 | 14308 | 6 | 6 | 6 | 7.43E−11 |
| 62 | PLCL2 | 23228 | 5760 | 5 | 5 | 5 | 7.89E−11 |
| 63 | LIFR | 3977 | 14497 | 6 | 6 | 6 | 8.03E−11 |
| 64 | SPEN | 23013 | 15178 | 6 | 6 | 6 | 1.05E−10 |
| 65 | KALRN | 8997 | 27441 | 7.5 | 9 | 8 | 1.27E−10 |
| 66 | MAGEC1 | 9947 | 5072 | 7.183824 | 32 | 20 | 1.37E−10 |
| 67 | LRP1B | 53353 | 34487 | 8 | 8 | 7 | 1.37E−10 |
| 68 | C16orf96 | 342346 | 6500 | 5 | 5 | 5 | 1.44E−10 |
| 69 | SMC2 | 10592 | 11297 | 10 | 10 | 5 | 1.44E−10 |
| 70 | C7orf58 | 79974 | 10383 | 6.025 | 46 | 41 | 1.53E−10 |
| 71 | KNTC1 | 9735 | 18824 | 7 | 7 | 6 | 1.85E−10 |
| 72 | AZU1 | 566 | 1907 | 4 | 4 | 4 | 1.93E−10 |
| 73 | RBM10 | 8241 | 6989 | 5 | 5 | 5 | 2.06E−10 |
| 74 | PCDHA2 | 56146 | 2818 | 4.25 | 8 | 8 | 2.08E−10 |
| 75 | CLCA4 | 22802 | 5937 | 6 | 9 | 7 | 2.25E−10 |
| 76 | MAST4 | 375449 | 17298 | 6 | 6 | 6 | 2.29E−10 |
| 77 | ATP2C2 | 9914 | 8224 | 6 | 6 | 5 | 2.32E−10 |
| 78 | ACTB | 60 | 2747 | 6 | 6 | 4 | 2.48E−10 |
| 79 | INPP5F | 22876 | 9298 | 6.043478 | 32 | 27 | 2.71E−10 |
| 80 | USH2A | 7399 | 34444 | 7 | 7 | 7 | 2.83E−10 |
| 81 | IGSF6 | 10261 | 2222 | 4 | 4 | 4 | 3.55E−10 |
| 82 | GPR98 | 84059 | 36956 | 11.085965 | 60 | 34 | 4.03E−10 |
| 83 | NPHP3 | 27031 | 1978 | 4.75 | 10 | 8 | 4.23E−10 |
| 84 | ZNF469 | 84627 | 13485 | 7.11014 | 70 | 52 | 4.42E−10 |
| 85 | CPSF1 | 29894 | 8236 | 5 | 5 | 5 | 4.66E−10 |
| 86 | TONSL | 4796 | 8248 | 5 | 5 | 5 | 4.70E−10 |
| 87 | FAN1 | 330554 | 8276 | 5 | 5 | 5 | 4.78E−10 |
| 88 | IQSEC2 | 23096 | 10549 | 5.25 | 9 | 9 | 4.78E−10 |
| 89 | APOB | 338 | 19621 | 6 | 6 | 6 | 4.83E−10 |
| 90 | RSF1 | 51773 | 8318 | 5 | 5 | 5 | 4.90E−10 |
| 91 | NBEA | 26960 | 22790 | 7 | 7 | 6 | 5.73E−10 |
| 92 | MIR205HG | 642587 | 1441 | 5.136364 | 38 | 26 | 5.74E−10 |
| 93 | ZFP36L1 | 677 | 3420 | 6 | 6 | 4 | 5.95E−10 |
| 94 | POLE | 5426 | 16635 | 6.2 | 11 | 10 | 6.14E−10 |
| 95 | DST | 667 | 38618 | 7 | 7 | 7 | 6.18E−10 |
| 96 | NVL | 4931 | 7367 | 4.833333 | 9 | 9 | 6.31E−10 |
| 97 | ZNFX1 | 57169 | 10057 | 6 | 6 | 5 | 6.31E−10 |
| 98 | FREM2 | 341640 | 20837 | 6 | 6 | 6 | 6.89E−10 |
| 99 | PCDHGA5 | 56110 | 2641 | 4 | 4 | 4 | 7.07E−10 |
| 100 | RECQL5 | 9400 | 9470 | 5.547619 | 28 | 25 | 7.70E−10 |
| 101 | MLL | 4297 | 23722 | 5.5 | 7 | 7 | 1.16E−08 |
| 102 | HRAS | 3265 | 2275 | 5 | 5 | 3 | 5.39E−08 |
| 103 | ERBB2 | 2064 | 10513 | 5 | 5 | 4 | 9.03E−08 |
| 104 | ERBB3 | 2065 | 11108 | 4.5 | 6 | 5 | 4.07E−07 |
| 105 | MLL3 | 58508 | 28340 | 10.65846 | 86 | 31 | 7.45E−07 |
| 107 | PI3KCA | 5290 | 7686 | 3 | 3 | 3 | 5.96E−06 |
| 108 | KRAS | 3845 | 6615 | 2.5 | 4 | 4 | 4.07E−05 |
| 106 | FGFR3 | 2261 | 7474 | 3.238095 | 26 | 23 | 8.22E−06 |
| 109 | TERT | 7015 | NA† | NA† | 2‡ | 24 | NA† |

*As noted above, the “gene length” for a particular diagnostic gene includes the length, in basepairs, of the exons of that diagnostic gene, plus 100 base pairs from each (e.g., 5′ and 3′) end of any intervening sequences (e.g., introns) within that diagnostic gene, plus 100 base pairs of the untranslated regions abutting the 5′-most and 3′-most ends of the coding region. This length was used in the gene weighting calculations of the studies to detect the diagnostic genes of the disclosure, as described above.
**Weighted Variant Count is as conducted by the gene “deweighting” procedure outlined above.
†NA = Not Applicable (weighting not conducted for TERT)
‡Only two mutations in the promoter of the TERT gene were assessed, C228T and C250T. Both involved C to T transitions, and are known “gain-of-function” mutations that result in the increased expression of the TERT gene product, telomerase reverse transcriptase.

### Example E

### GENE LIST X

Also provided is a 61-member subset of the genes of GENE LIST C, known as GENE LIST X. GENE LIST X comprises AHNAK2, AKAP13, BTN2A2, CARD6, CCL20, CLCA4, COL12A1, COL21A1, CPSF2, DCHS1, DNAH17, DNAJC10, DOCKS, DYNC1H1, FAM193A, FLG, GLDC, HMCN1, HSPBAP1, IGSF6, ISG20L2, ITGA8, JMJD1C, KDM6A, KIAA0100, KIAA1671, KPRP, LYST, MFHAS1, MLL2, MYBPC2, NCKAP1L, NPC1L1, NPHP3, NUP188, ODZ3, PCLO, PDE4A, PLCG2, PLXNA2, PSD4, RBBP8, SAMD4A, SCN9A, SMC2, SNRNP200, SPAG17, STAG2, SYNE1, TNP2, UGGT1, UGT1A3, VCX3B, VPS13D, WDR6, XIRP2, XPOT, ZC3H7A, ZFHX4, ZNF208, and ZNF493. The bladder cancer-specific signature mutations identified in the genes of GENE LIST X are provided within MUTATION PANELS B and C. Additional information about the members of GENE LIST X is presented below, in Table 11.

TABLE 11
Diagnostic genes bearing bladder cancer-specific signature mutations
(GENE LIST X)

| | Entrez | Gene |
|---|---|---|
| Gene | Gene ID | Length* |
| AHNAK2 | 113146 | 19572 |
| AKAP13 | 11214 | 21025 |
| BTN2A2 | 10385 | 5367 |
| CARD6 | 84674 | 4694 |
| CCL20 | 6364 | 1638 |
| CLCA4 | 22802 | 5937 |
| COL12A1 | 81578 | 24090 |
| COL21A1 | 81578 | 9746 |
| CPSF2 | 53981 | 8135 |
| DCHS1 | 8642 | 14137 |
| DNAH17 | 8632 | 27298 |
| DNAJC10 | 54431 | 8969 |
| DOCK9 | 23348 | 19186 |
| DYNC1H1 | 1778 | 27420 |
| FAM193A | 8603 | 8372 |
| FLG | 2312 | 13344 |
| GLDC | 2731 | 8759 |
| HMCN1 | 83872 | 39142 |
| HSPBAP1 | 79663 | 3455 |
| IGSF6 | 10261 | 2222 |
| ISG20L2 | 81875 | 2648 |
| ITGA8 | 8516 | 9231 |
| JMJD1C | 221037 | 13810 |
| KDM6A | 7403 | 11359 |
| KIAA0100 | 9703 | 13143 |
| KIAA1671 | 85379 | 12683 |
| KPRP | 448834 | 2921 |
| LYST | 1130 | 23851 |
| MFHAS1 | 9258 | 5852 |
| MLL2 | 8085 | 27398 |
| MYBPC2 | 4606 | 8698 |
| NCKAP1L | 3071 | 9933 |
| NPC1L1 | 29881 | 8897 |
| NPHP3 | 27031 | 1978 |
| NUP188 | 23511 | 13570 |
| ODZ3 | 55714 | 16176 |
| PCLO | 27445 | 27278 |
| PDE4A | 5141 | 8907 |
| PLCG2 | 5336 | 10808 |
| PLXNA2 | 5362 | 17765 |
| PSD4 | 23550 | 8261 |
| RBBP8 | 5932 | 7416 |
| SAMD4A | 23034 | 9419 |
| SCN9A | 6335 | 15069 |
| SMC2 | 10592 | 11297 |
| SNRNP200 | 23020 | 15227 |
| SPAG17 | 200162 | 16454 |
| STAG2 | 10735 | 13183 |
| SYNE1 | 23345 | 57157 |
| TNP2 | 7142 | 995 |
| UGGT1 | 56886 | 19121 |
| UGT1A3 | 54659 | 1066 |
| VCX3B | 425054 | 1764 |
| VPS13D | 55187 | 30041 |
| WDR6 | 11180 | 4895 |
| XIRP2 | 129446 | 15610 |
| XPOT | 11260 | 8684 |
| ZC3H7A | 29066 | 8286 |
| ZFHX4 | 79776 | 16147 |
| ZNF208 | 7757 | 9884 |
| ZNF493 | 284443 | 7280 |

*As noted above, the “gene length” for a particular diagnostic gene includes the length, in basepairs, of the exons of that diagnostic gene, plus 100 base pairs from each (e.g., 5′ and 3′) end of any intervening sequences (e.g., introns) within that diagnostic gene, plus 100 base pairs of the untranslated regions abutting the 5′-most and 3′-most ends of the coding region. This length was used in the gene weighting calculations of the studies to detect the diagnostic genes of the disclosure, as described above.

### Example F

### Urine Sample Preparation

Urine samples to be used in the diagnostic methods of the present disclosure can be collected, contained and stored as commonly practiced in the medical arts. However, it is important that steps are taken to protect the DNA present in the urine from degradation, whether that DNA is present in the form of chromatin in the nuclei of nucleated cells in the urine, or is cell-free DNA. Consequently, stabilizing agents can be added to a defined volume of freshly-collected urine before the urine sample is stored prior to analysis. Such stabilizing agents to be added to a measured volume (e.g., 10 mL or 50 mL) of freshly-collected urine can include, for example, reagents in dry or dissolved liquid form, such as ethylenediaminetetraacetic acid (EDTA) or other preservatives, protease inhibitors such as proteinase K, nuclease inhibitors, antimicrobial agents such as sodium azide, buffers, salts, etc.

Ideally DNA is obtained from the stabilized urine sample immediately after collection and stabilization. However, stabilized urine samples can also be stored, preferably under refrigeration at 4° C., or frozen at −20° C. or −80° C., until such time as the DNA can be obtained from the sample.

If it is known that the DNA to be obtained from a urine sample is to be obtained from nucleated cells in the sample, reagents designed to stabilize intact cells within the urine can also be added. An example of a reagent especially designed to stabilize intact cells in urine are the Stabilur® tablets available from Cargille Laboratories (Cedar Grove, N.J.; Product No. 40050). A single Stabilur® tablet is used to stabilize cells in 10 mL of urine prior to processing, and the sample should either process immediately or stored at room temperature for no more than 72 h. Such samples should not be frozen, as the process of freezing and thawing results in undesired lysis of cells.

### Example G

### Isolation of Cellular DNA from Urine

When the DNA that is obtained from a urine sample is to be obtained from nucleated cells present in the urine, the cells may first be isolated from the urine sample by sedimentation (through centrifugation) or filtration. For example, after mixing, collected urine samples can be aliquoted in 50 mL portions for further processing. Each 50 mL can then be centrifuged at 3000×g for 10 min. to pellet the cells. The cells in the cell pellet can then be lysed by the addition of a suitable detergent, and DNA can be extracted from the lysate.

Extraction of genomic DNA from nucleated cells can be conducted using methods known in the art, or by using commercially available products for this purpose. For example, the DNA in pelleted nucleated cells can be extracted using the Gentra Puregene® Cell Kit from Qiagen, Inc. (Valencia, Calif.), according to manufacturer's instructions. Genomic DNA so obtained can be resuspended an appropriate buffer, and stored under refrigeration prior to analysis. The DNA so obtained can be quantified using any suitable method, so that the volume of resuspended genomic DNA required for mutation analysis can be readily determined.

Detailed methods for the separate isolation of cellular DNA and cell-free DNA from urine that can be used for the methods of the present disclosure are described in Beermann, A. et al. “Methods for Separate Isolation of Cell-Free DNA and Cellular DNA from Urine—Application of Methylation-Specific PCR on both DNA Fractions.” The Open Biomarkers Journal (2011) 4:15-17. Other methods for isolating DNA from urine are described in Deelman, L. E., et al. “A Method for the Ultra Rapid Isolation of PCR-Ready DNA from Urine and Buccal Swabs.” Molecular Biology Today (2002) 3:51-54.

### Example H

### Isolation of Cell-Free DNA from Urine

If the DNA that is obtained from a urine sample and used to determine the present of an indicator of bladder cancer is cell-free DNA, it may be isolated by ultrafiltration of the urine, or by passing the urine through a cationic matrix that binds the negatively-charged DNA, or an appropriate device that contains such a cationic matrix.

Obtaining cell-free DNA from urine samples can be accomplished using methods known in the art, or by using commercially available products for this purpose. For example, the cell-free DNA in urine can be extracted using the Urine DNA Isolation Micro Kit available from Norgen (Thorold, Ontario, Canada; Catalog No. 18100) according to the manufacturer's instructions. Cell-free DNA so obtained can be eluted an appropriate buffer, and stored under refrigeration prior to analysis. The DNA so obtained can be quantified using any suitable method, so that the volume of eluted DNA required for mutation analysis can be readily determined.

Detailed methods for the separate isolation of cellular DNA and cell-free DNA from urine that can be used for the methods of the present disclosure are described in Beermann et al. “Methods for Separate Isolation of Cell-Free DNA and Cellular DNA from Urine—Application of Methylation-Specific PCR on both DNA Fractions.” The Open Biomarkers Journal (2011) 4:15-17. Other methods for isolating DNA from urine are described in Deelman, L. E., et al. “A Method for the Ultra Rapid Isolation of PCR-Ready DNA from Urine and Buccal Swabs.” Molecular Biology Today (2002) 3:51-54.

### Example I

### Analysis of DNA Isolated from Urine

As noted above, for the methods of the present disclosure, the determining step comprises detecting the presence of one or more indicators of bladder cancer, including detecting one or more of the tumor-specific signature mutations in a one or more of the diagnostic genes in Table 6 or Table 4, or in a particular diagnostic test panel. For all of such methods, the detecting of mutations can be accomplished by any suitable method used in the art. For example, the detecting step may involve detection by a technique chosen from allele-specific polymerase chain reactions (PCR), mutant-enriched PCR, digital protein truncation tests, direct sequencing, massively parallel sequencing, use of molecular beacon probes or primers, BEAMing digital PCR, or allele-specific hybridization. These techniques are well known in the art and have been described in detail in printed publications, laboratory protocol manuals, and technical literature provided by the manufacturers of the reagents and instruments used to conduct them. Exemplary protocols and a comparison of available methods can be found, for example, in the following books, book chapters and references:
Theophilus, B. D. M. (ed.) and Rapley, R. (ed.); PCR Mutation Detection Protocols; 2nd Edition; (2011); Series: Methods in Molecular Biology; 305 p.; Humana Press;
Taylor, C. F. and Taylor, G. R.; “Current and Emerging Techniques for Diagnostic Mutation Detection: An Overview of Methods for Mutation Detection;” Chapter 2; Molecular Diagnosis of Genetic Diseases; (2003); Series: Methods in Molecular Medicine, Vol. 92; Pages 9-44; Springer Protocols—Springer Press;
Edwards, J. and Bartlett, J. M. S.; “Mutation and Polymorphism Detection: A Technical Overview;” Chapter 41; PCR Protocols; (2003); Series: Methods in Molecular Biology, Vol. 226; Pages 287-293; Springer Protocols—Springer Press;
Taylor, G. R. (ed.); Laboratory Methods for the Detection of Mutations and Polymorphisms in DNA; (1997); 333 p.; CRC Press;
Diehl, F., et al.; “Circulating Mutant DNA to Assess Tumor Dynamics;” Nature Medicine; (2008) 14:985-990.

Additionally, methods specific to identifying single-nucleotide polymorphisms in the genomes of bladder cancers have been previously described, and can be adapted for use in the methods of the present disclosure, using the tumor-specific signature mutations identified herein. These methods are described, for example, in the following references:
Hogue, M. O., et al.; “Genome-Wide Genetic Characterization of Bladder Cancer: A Comparison of High-Density Single-Nucleotide Polymorphism Arrays and PCR-based Microsatellite Analysis;” Cancer Res.; (2003) 63:2216-2222; and
Hogue, M. O., et al.; “High-Throughput Molecular Analysis of Urine Sediment for the Detection of Bladder Cancer by High-Density Single-Nucleotide Polymorphism Array;” Cancer Res.; (2003) 63:5723-5726.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing disclosure has been described in some detail by way of illustration and example for purposes of clarity of understanding, it will be obvious that certain changes and modifications may be practiced within the scope of the appended claims.

1. An in vitro method of detecting bladder cancer, comprising:
(1) analyzing nucleic acid (e.g., DNA) in or obtained from a patient biological sample (e.g., urine) to detect the presence or absence of at least one indicator of bladder cancer in said nucleic acid, wherein said indicator is a mutation (e.g., a signature mutation listed in Table A, B, C, or D, or another missense mutation, nonsense mutation, frameshift mutation, splicing mutation, or large rearrangement, or a combination thereof) in one or more genes listed in Table 6 or Table 4; and
(2)(a) diagnosing a patient in whose sample the presence of said indicator of bladder cancer is detected as having bladder cancer (or as having a high risk of having bladder cancer); or
optionally (2)(b) diagnosing a patient in whose sample the absence of any indicator of bladder cancer is detected as not having bladder cancer (or as having a low risk of having bladder cancer).

2. The method of claim 1, wherein said indicator is a signature mutation listed in Table A, B, C, or D.

3. The method of claim 1, wherein said indicator is a missense mutation, nonsense mutation, insertion, or deletion in the coding sequence for the protein encoded by one of the genes listed in Table 6 or Table 4.

4. The method of claim 1, wherein said indicator is a mutation that alters the splicing of the transcript of one of the genes listed in Table 6 or Table 4.

5. The method of claim 1, wherein the presence (or optionally the absence) of a plurality of signature mutations listed in Table A, B, C, or D is detected.

6. The method of claim 1, wherein the presence of (or optionally the absence) of a plurality of missense mutations, nonsense mutations, insertions or deletions, or transcript splicing altering mutations, or any combination thereof, is detected.

7. The method of claim 1 wherein said bladder cancer is a recurrent bladder cancer.

8. The method of claim 1, further comprising obtaining nucleic acid from a patient biological sample, wherein said obtaining comprises the steps of:
(a) isolating cells from a urine sample from said patient;
(b) lysing the cells; and
(c) extracting the nucleic acid from the cell lysate.

9. The method of claim 8, wherein the isolating step comprises isolating the cells by centrifugation or filtration of the urine sample.

10. The method of claim 8, wherein the lysing step comprises treating the isolated cells with a detergent or surfactant to provide a lysate.

11. The method of claim 8, wherein the extracting step comprises treating the cell lysate with a nucleic acid extraction reagent and concentrating the extracted nucleic acid by precipitation or by binding it to a cationic matrix.

12. The method of claim 1, wherein the nucleic acid analyzed in step (1) is cell-free nucleic acid isolated from a urine sample.

13. The method of claim 12, further comprising the step of obtaining nucleic acid from a patient biological sample, wherein said obtaining comprises isolating cell-free nucleic acid from a urine sample and wherein isolating cell-free nucleic acid from a urine sample comprises passing the urine sample through a cationic matrix that binds the cell-free nucleic acid or concentrating the cell-free nucleic acid by ultrafiltration of the urine sample.

14. The method of claim 1, wherein step (1) comprises detecting the presence or absence of one or more mutations that are the signature mutations listed in Table A, B, C, or D.

15. The method of claim 1, wherein step (1) comprises detecting the presence or absence of one or more mutations that is a missense mutation, nonsense mutation, insertion, or deletion in the coding sequence for the protein encoded by one of the genes listed in Table 6 or Table 4.

16. The method of claim 1, wherein step (1) comprises detecting the presence or absence of one or more mutations that is a mutation that alters the splicing of the transcript of one of the genes listed in Table 6 or Table 4.

17. The method of claim 1, wherein the one or more mutations are detected by a technique chosen from allele-specific PCR, mutant-enriched PCR, digital protein truncation test, direct sequencing, massively parallel sequencing, use of molecular beacon probes or primers, BEAMing digital PCR, or allele-specific hybridization.

18. The method of claim 1, further comprising (4) generating a risk profile using the results of steps (1), (2) and (3).

19. The method of claim 1, wherein said bladder cancer is selected from the group consisting of premalignant and malignant bladder cancer.

20. The method of claim 1, wherein said method permits detection of bladder cancer in said human patient with a sensitivity of at least 85% at a specificity of at least 85%, with a sensitivity of at least 80% at a specificity of at least 90%, with a sensitivity of at least 75% at a specificity of at least 95%, or with a sensitivity of at least 70% at a specificity of at least 95%.

21-86. (canceled)
